(19) United States
(12) Patent Application Publication
(10) Pub. No.: US 2016/0250224 A1
(43) Pub. Date: Sep. 1, 2016

(54) OREXIN-CONTROL OF BONE FORMATION AND LOSS

(71) Applicant: THE BOARD OF REGENTS OF THE UNIVERSITY OF TEXAS SYSTEM, Austin, TX (US)

(72) Inventor: Yihong WAN, Coppell, TX (US)

(73) Assignee: The Board of Regents of the University of Texas System, Austin, TX (US)

(21) Appl. No.: 15/024,354

(22) PCT Filed: Sep. 24, 2014
(86) PCT No.: PCT/US14/57156

§ 371 Date: Mar. 23, 2016

Related U.S. Application Data
(60) Provisional application No. 61/881,715, filed on Sep. 24, 2013.

(51) Int. Cl. A61K 31/551; A61K 31/472; A61K 9/00; A61K 31/454

(52) U.S. Cl. CPC A61K 31/551; A61K 31/454; A61K 31/472; A61K 9/0019

(57) ABSTRACT

The use of orexin 1 receptor antagonists and/or orexin 2 receptor agonists thereof in the treatment of bone loss diseases is described. Such conditions include osteoporosis, rheumatoid arthritis and other bone wasting diseases.

This application claims the benefit of priority to U.S. Provisional Application Ser. No. 67/881,715, filed Sep. 24, 2013, the entire contents of which are hereby incorporated by reference.

The invention was made with government support under Grant No. R01DK089113 awarded by the National Institutes of Health. The government has certain rights in the invention.

### BACKGROUND OF THE INVENTION

1. Field of the Invention

The present invention relates generally to the fields of medicine, pathology and molecular biology. More particularly, it concerns the involvement of Orexin receptor 1 and 2 function in the regulation of bone formation and loss. Specifically, the invention relates to the use of antagonists and agonists of these receptors for treating bone loss.

2. Description of Related Art

Orexin-A and -B (also known as hypocretin-1 and -2) are neuropeptides produced in the lateral hypothalamus that stimulate wakefulness, feeding, thermogenesis and reward behaviors (Sakurai, 2007; Sakurai and Mieda, 2011). They function through two receptors: OX1R and OX2R. Orexin deficiency in human and mice leads to narcolepsy, hypophagia and obesity (Chemelli et al., 1999; Hara et al., 2001; Lin et al., 1999; Peyron et al., 2000; Sellayah et al., 2011). Hence, there is tremendous pharmacological interest in developing orexin-targeting small molecules for the treatment of sleep and metabolic disorders such as insomnia (Brisbare-Roch et al., 2007), obesity and diabetes (Funato et al., 2009; Kotz et al., 2012; Sellayah et al., 2011), some of which has completed Phase III clinical trials.

In vertebrates, the adult skeleton is continuously regenerated through bone remodeling. This is a dynamic process that tightly couples osteoblast-mediated bone formation with osteoclast-mediated bone resorption. Osteoblasts are derived from bone marrow mesenchymal stem cells (MSCs) that can also differentiate into marrow adipocytes, the balance of which is controlled by an array of hormones and transcription factors (Bianco et al., 2013; Wan, 2013). In contrast, osteoclasts are differentiated from macrophage precursors in response to Receptor Activator of NFκB Ligand (RANKL), depending on the ratio of RANKL to OPG (osteoprotegerin), a RANKL decoy receptor that inhibits osteoclast differentiation (Novack and Teitelbaum, 2008).

Emerging evidence reveal that neuropeptides, such as neuromedin U (NMU) and neuropeptide Y (NPY), modulate skeletal homeostasis via both central and peripheral functions (Rosen, 2008). However, whether orexin regulates bone mass accrual is unknown. This is an important question in light of the therapeutic potential of orexin modulators in several diseases.

### SUMMARY OF THE INVENTION

Thus, in accordance with the present disclosure, there is provided a method of increasing bone mass and/or volume in a subject comprising (a) identifying a patient in need of increased bone mass and/or volume; and (b) administering to said subject an agonist of orexin receptor 2 and/or an antagonist of orexin receptor 1. The agonist and/or antagonist may be administered intravenously, intra-peritoneally, intramuscularly, subcutaneously or topically. The agonist and/or antagonist may be administered to a bone target site, such as injected at said site. The agonist and/or antagonist may be comprised in a time-release device implanted at said site. The subject may be a human or a non-human animal, such as a mouse, a rat, a rabbit, a dog, a cat, a horse, a monkey or a cow. The method may further comprise at least a second administration of said agonist and/or antagonist, such as at least three administrations per week, or at least 12 administrations in total. The method may further comprise assessing bone mass following administration of said agonist or antagonist, such as by bone imaging. The subject may suffer from osteoporosis, bone fracture, bone loss due to trauma, rheumatoid arthritis or Paget's Disease. The subject may be one who does not have one or more of insomnia, diabetes, obesity, migraine, cluster headache, Parkinson's disease, Alzheimer's disease, depression, addictions, anxiety, cancer, irritable bowel syndrome, narcolepsy, neuropathic pain, pain, schizophrenia, sleep disorder, Tourette syndrome, Crushings syndrome, gonadotropinoma, gastrinoma, Zollinger-Ellison syndrome, hypersecretory diarrhea related to AIDS and other conditions, irritable bowel syndrome, pancreatitis, Crohn's disease, systemic sclerosis, thyroid cancer, psoriasis, hypotension, panic attacks, scleroderma, small bowel obstruction, gastroesophageal reflux, duodenogastric reflux, Grave's disease, polycystic ovary disease, upper gastrointestinal bleeding, pancreatic pseudocyst, pancreatic ascites, leukemia, meningioma, cancer cachexia, acromegaly, restenosis, hepatoma, lung cancer, melanoma, wasting, type 2 diabetes, Syndrome X, fibrosis, hyperlipidemia, hyperamylinemia, hyperprolactinemia, prolactinomas, cluster headache, depression, neuropathic pain or pain.

In another embodiment, there is provided a method of increasing bone growth in a subject comprising (a) identifying a patient in need of increased bone growth; and (b) administering to said subject an agonist of orexin receptor 2 and/or an antagonist of orexin receptor 1. The agonist and/or antagonist may be administered intravenously, intra-peritoneally, intramuscularly, subcutaneously or topically. The agonist and/or antagonist may be administered to a bone target site, such as injected at said site. The agonist and/or antagonist may be comprised in a time-release device implanted at said site. The subject may be a human or a non-human animal, such as a mouse, a rat, a rabbit, a dog, a cat, a horse, a monkey or a cow. The method may further comprise at least a second administration of said agonist and/or antagonist, such as at least three administrations per week, or at least 12 administrations in total. The method may further comprise assessing bone mass following administration of said agonist or antagonist, such as by bone imaging. The subject may suffer from osteoporosis, bone fracture, bone loss due to trauma, rheumatoid arthritis or Paget's Disease. The subject may be one who does not have one or more of insomnia, diabetes, obesity, migraine, cluster headache, Parkinson's disease, Alzheimer's disease, depression, addictions, anxiety, cancer, irritable bowel syndrome, narcolepsy, neuropathic pain, pain, schizophrenia, sleep disorder, Tourette syndrome, Crushings syndrome, gonadotropinoma, gastrinoma, Zollinger-Ellison syndrome, hypersecretory diarrhea related to AIDS and other conditions, irritable bowel syndrome, pancreatitis, Crohn's disease, systemic sclerosis, thyroid cancer, psoriasis, hypotension, panic attacks, scleroderma, small bowel obstruction, gastroesophageal reflux, duodenogastric reflux, Grave's disease, polycystic ovary disease, upper gastrointestinal bleeding, pancreatic pseudocyst, pancreatic ascites, leukemia, meningioma, cancer cachexia, acromegaly, restenosis, hepatoma, lung cancer, melanoma, wasting, type 2 diabetes, Syndrome X, fibrosis, hyperlipidemia, hyperamylinemia, hyperprolactinemia, prolactinomas, cluster headache, depression, neuropathic pain or pain.

In yet another embodiment, there is provided a method of increasing osteoblast number in a subject comprising (a) identifying a patient in need of increased osteoblast number; and (b) administering to said subject an agonist of orexin receptor 2. The agonist may be administered intravenously, intra-peritoneally, intramuscularly, subcutaneously or topically. The agonist may be administered to a bone target site, such as injected at said site. The agonist may be comprised in a time-release device implanted at said site. The subject may be a human or a non-human animal, such as a mouse, a rat, a rabbit, a dog, a cat, a horse, a monkey or a cow. The method may further comprise at least a second administration of said agonist, such as at least three administrations per week, or at least 12 administrations in total. The method may further comprise assessing bone mass following administration of said agonist, such as by bone imaging. The subject may suffer from osteoporosis, bone fracture, bone loss due to trauma, rheumatoid arthritis or Paget's Disease. The subject may be one who does not have one or more of insomnia, diabetes, obesity, migraine, cluster headache, Parkinson's disease, Alzheimer's disease, depression, addictions, anxiety, cancer, irritable bowel syndrome, narcolepsy, neuropathic pain, pain, schizophrenia, sleep disorder, Tourette syndrome, Crushings syndrome, gonadotropinoma, gastrinoma, Zollinger-Ellison syndrome, hypersecretory diarrhea related to AIDS and other conditions, irritable bowel syndrome, pancreatitis, Crohn's disease, systemic sclerosis, thyroid cancer, psoriasis, hypotension, panic attacks, scleroderma, small bowel obstruction, gastroesophageal reflux, duodenogastric reflux, Grave's disease, polycystic ovary disease, upper gastrointestinal bleeding, pancreatic pseudocyst, pancreatic ascites, leukemia, meningioma, cancer cachexia, acromegaly, restenosis, hepatoma, lung cancer, melanoma, wasting, type 2 diabetes, Syndrome X, fibrosis, hyperlipidemia, hyperamylinemia, hyperprolactinemia, prolactinomas, cluster headache, depression, neuropathic pain or pain.

In a further embodiment, there is provided a method of decreasing osteoclast activity in a subject comprising (a) identifying a patient in need of decreased osteoclast activity; and (b) administering to said subject an antagonist of orexin receptor 1. The antagonist may be administered intravenously, intra-peritoneally, intramuscularly, subcutaneously or topically. The antagonist may be administered to a bone target site, such as injected at said site. The antagonist may be comprised in a time-release device implanted at said site. The subject may be a human or a non-human animal, such as a mouse, a rat, a rabbit, a dog, a cat, a horse, a monkey or a cow. The method may further comprise at least a second administration of said antagonist, such as at least three administrations per week, or at least 12 administrations in total. The method may further comprise assessing bone mass following administration of said antagonist, such as by bone imaging. The subject may suffer from osteoporosis, bone fracture, bone loss due to trauma, rheumatoid arthritis or Paget's Disease. The subject may be one who does not have one or more of insomnia, diabetes, obesity, migraine, cluster headache, Parkinson's disease, Alzheimer's disease, depression, addictions, anxiety, cancer, irritable bowel syndrome, narcolepsy, neuropathic pain, pain, schizophrenia, sleep disorder, Tourette syndrome, Crushings syndrome, gonadotropinoma, gastrinoma, Zollinger-Ellison syndrome, hypersecretory diarrhea related to AIDS and other conditions, irritable bowel syndrome, pancreatitis, Crohn's disease, systemic sclerosis, thyroid cancer, psoriasis, hypotension, panic attacks, scleroderma, small bowel obstruction, gastroesophageal reflux, duodenogastric reflux, Grave's disease, polycystic ovary disease, upper gastrointestinal bleeding, pancreatic pseudocyst, pancreatic ascites, leukemia, meningioma, cancer cachexia, acromegaly, restenosis, hepatoma, lung cancer, melanoma, wasting, type 2 diabetes, Syndrome X, fibrosis, hyperlipidemia, hyperamylinemia, hyperprolactinemia, prolactinomas, cluster headache, depression, neuropathic pain or pain.

In yet a further embodiment, there is provodied a method of increasing bone strength in a subject comprising (a) identifying a patient in need of increased bone strength; and (b) administering to said subject an agonist of orexin receptor 2 and/or an antagonist of orexin receptor 1. The agonist and/or antagonist may be administered intravenously, intraperitoneally, intramuscularly, subcutaneously or topically. The agonist and/or antagonist may be administered to a bone target site, such as injected at said site. The agonist and/or antagonist may be comprised in a time-release device implanted at said site. The subject may be a human or a non-human animal, such as a mouse, a rat, a rabbit, a dog, a cat, a horse, a monkey or a cow. The method may further comprise at least a second administration of said agonist and/or antagonist, such as at least three administrations per week, or at least 12 administrations in total. The method may further comprise assessing bone mass following administration of said agonist or antagonist, such as by bone imaging. The subject may suffer from osteoporosis, bone fracture, bone loss due to trauma, rheumatoid arthritis or Paget's Disease. The subject may be one who does not have one or more of insomnia, diabetes, obesity, migraine, cluster headache, Parkinson's disease, Alzheimer's disease, depression, addictions, anxiety, cancer, irritable bowel syndrome, narcolepsy, neuropathic pain, pain, schizophrenia, sleep disorder, Tourette syndrome, Crushings syndrome, gonadotropinoma, gastrinoma, Zollinger-Ellison syndrome, hypersecretory diarrhea related to AIDS and other conditions, irritable bowel syndrome, pancreatitis, Crohn's disease, systemic sclerosis, thyroid cancer, psoriasis, hypotension, panic attacks, scleroderma, small bowel obstruction, gastroesophageal reflux, duodenogastric reflux, Grave's disease, polycystic ovary disease, upper gastrointestinal bleeding, pancreatic pseudocyst, pancreatic ascites, leukemia, meningioma, cancer cachexia, acromegaly, restenosis, hepatoma, lung cancer, melanoma, wasting, type 2 diabetes, Syndrome X, fibrosis, hyperlipidemia, hyperamylinemia, hyperprolactinemia, prolactinomas, cluster headache, depression, neuropathic pain or pain.

The use of the word “a” or “an” when used in conjunction with the term “ comprising” in the claims and/or the specification may mean “one,” but it is also consistent with the meaning of “one or more,” “at least one,” and “one or more than one.”

It is contemplated that any embodiment discussed herein can be implemented with respect to any method or composition of the invention, and vice versa. Furthermore, compositions and kits of the invention can be used to achieve methods of the invention.

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The invention may be better understood by reference to one or more of these drawings in combination with the detailed description of specific embodiments presented herein.

FIGS. 1A-L. Orexin Deletion Causes Low-Bone-Mass and Decreased Bone Formation. (FIGS. 1A-E) μCT analysis of tibiae from OX-KO or WT controls (3 month old, male, n=9). (FIG. 1A) Images of the trabecular bone of the tibial metaphysis (top) (scale bar, 10 μm) and the entire proximal tibia (bottom) (scale bar, 1 mm) (FIGS. 1B-D) Trabecular bone parameters. (FIG. 1B) BV/TV, bone volume/tissue volume ratio; Tb.N, trabecular number; Tb.Th, trabecular thickness; Tb.Sp, trabecular separation. (FIG. 1C) BMD, bone mineral density. (FIG. 1D) SMI, Structure Model Index. (FIG. 1E) Additional trabecular and cortical bone parameters. BS, bone surface; BS/BV, bone surface/bone volume; Tb. Porosity, trabecular bone porosity. (FIG. 1F) Three-point bending assay (n=9). (FIG. 1G) Serum P1NP (n=9). (FIG. 1H) Serum CTX-1 (n=9). (FIGS. 1I-J) Static histomorphometry of osteoblast number (Ob.N/B.Ar) (FIG. 1I) and osteoclast number (Oc.N/B.Ar) (FIG. 1J) (n=9); B.Ar, bone area. (FIGS. 1K-M) Dynamic histomorphometry (3 month old at end point, male, n=5). (FIG. 1K) Images of the trabecular bone at metaphysis and the cortical bone at diaphysis. Scale bars, 10 μm. (FIG. 1L) Bone formation rate (BFR/BS). (FIG. 1M) Mineral apposition rate (MAR). Error bars, SD.

FIGS. 2A-Z. OX1R Decreases Bone Mass via a Local Regulation of Bone Cell Differentiation. (FIG. 2A) Left, expression of orexin, OX1R and OX2R in mouse tibiae (bone+marrow) (n=3); n.d., not detected; Right, orexin expression in various cell types (n=10): MSC, mesenchymal stem cell; Ob, osteoblast; Ad, marrow adipocyte; mf, macrophage; Oc, osteoclast. (FIG. 2B) Expression of OX1R and OX2R in marrow osteoblast differentiation cultures (n=3). ObDiff, osteoblast differentiation cocktail. (FIG. 2C) Expression of OX1R during a time course of osteoblast differentiation (n=3); d, day. (FIG. 2D) Expression of OX1R and OX2R in marrow adipocyte differentiation cultures (n=3). AdDiff, adipocyte differentiation medium. (FIG. 2E) Expression of OX1R during a time course of adipocyte differentiation (n=3). (FIGS. 2F-G) Effects of OX-A or OX-B (10 nM) on osteoblast (FIG. 2F) and adipocyte (FIG. 2G) differentiation from WT marrow (n=3). (FIGS. 2H-J) Effects of OX1R inhibitor (OX1R-I), OX2R inhibitor (OX2R-I) or OX1R2R dual inhibitor (1R2R-I) (10 nM) on osteoblast (FIG. 2H), adipocyte (FIG. 2I) and osteoclast (FIG. 2J) differentiation from WT marrow (n=3). (FIGS. 2K-O) μCT analysis of tibiae from OX1R-KO or WT controls (3 month old, male, n=9). (FIG. 2K) Representative images. (FIGS. 2L-O) Trabecular and cortical bone parameters. (FIG. 2P) Three-point bending assay (n=9). (FIG. 2Q) Serum P1NP (left) and CTX-1 (right) (n=9). (FIG. 2R) Static histomorphometry (n=9). (FIG. 2S) Dynamic histomorphometry of the trabecular bone at metaphysis (3 month old, male, n=5). (T-U) Osteoblast differentiation from the bone marrow of OX1R-KO or WT control mice (n=3). (FIG. 2T) Alkaline phosphatase (ALP, top), alizarin red (middle) or von Kossa (bottom) stained osteoblast differentiation cultures. (FIG. 2U) Expression of osteoblast markers (n=3). (FIGS. 2V-W) Adipocyte differentiation from the bone marrow of OX1R-KO or WT control mice (n=3). (FIG. 2V) Oil Red O (ORO) stained adipocyte differentiation culture. Scale bar, 100 μm. (FIG. 2W) Expression of adipocyte markers (n=3). (FIG. 2X) Osteoclast differentiation quantified by TRAP expression (n=3). (FIGS. 2Y-Z) Expression of RANKL (FIG. 2Y) and OPG (FIG. 2Z) in the osteoblast differentiation culture (n=3). “+” and “n.s.” in F-I compares treatment with vehicle control; “+” and “n.s.” in U and W-Z compares OX1R-KO with WT control under the same treatment conditions. Error bars, SD.

FIGS. 3A-Z. OX2R Increases Bone Mass via a Central Regulation. (FIGS. 3A-E) μCT of tibiae from OX2R-KO or WT controls (3 month old, male, n=9). (FIG. 3A) Representative images. (FIGS. 3B-E) Trabecular and cortical bone parameters. (FIG. 3F) Three-point bending assay (n=9). (FIG. 3G) Serum P1NP (n=9). (FIG. 3H) Serum CTX-1 (n=9). (FIG. 3I) Static histomorphometry (n=9). (FIG. 3J) Dynamic histomorphometry of the trabecular bone at metaphysis and the cortical bone at diaphysis (3 month old, male, n=5). (FIGS. 3K-L) Osteoblast differentiation (FIG. 3K) and adipocyte differentiation (FIG. 3L) from the marrow of OX2R-KO mice and WT controls (n=3). (FIGS. 3M-T) Effects of ICV injection of an OX2R agonist (OX2R-AG) on bone in 12-week-old WT male mice (n=5). (FIGS. 3M-Q) μCT analysis of tibiae. (FIG. 3M) Representative images. (FIGS. 3N-Q) Trabecular and cortical bone parameters. (FIG. 3R) Three-point bending assay. (FIG. 3S) Serum P1NP. (FIG. 3T) Serum CTX-1. (FIGS. 3U-Z) Effects of ICV injection of an OX2R-AG on OVX-induced bone loss in 6-month-old WT female mice (n=4). (FIG. 3U) Uterine weight. (FIG. 3V) Serum P1NP. (FIG. 3W) Serum CTX-1. (FIGS. 3X-Y) μCT and histomorphometry. (FIG. 3X) (top) μCT images; (bottom) histomorphometry analyses. p<0.05 by ANOVA; *compares “OVX+Veh” with “Sham+Veh”; ⁺ compares “OVX+OX2R-AG ” with “OVX+Veh”. (FIG. 3Y) Trabecular bone parameters. (FIG. 3Z) Three-point bending assay. “n.s.” in K-L compares OX2R-KO with WT control under the same treatment conditions. (FIGS. 3U-Z) Statistical analysis was performed by ANOVA and the post-hoc Tukey pairwise comparisons. Error bars, SD.

FIGS. 4A-T. Central Action is Dominant over Peripheral Action in Orexin Regulation of Bone. (FIGS. 4A-J) Analysis of 1R2R-DKO mice and WT controls (3 month old, male, n=9). (FIGS. 4A-E) μCT of tibiae. (A) Representative images. (FIGS. 4B-E) Trabecular and cortical bone parameters. (FIG. 4F) Three-point bending assay. (FIG. 4G) Serum P1NP. (FIG. 4H) Serum CTX-1. (FIG. 4I) Static histomorphometry. (FIG. 4J) Dynamic histomorphometry (3 month old, male, n=5). (FIGS. 4K-T) Analysis of OX-Tg mice (n=10) and WT littermate controls (n=8) (3-4 month old, male). (FIGS. 4K-O) μCT of tibiae. (FIG. 4K) Representative images. (FIGS. 4L-O) Trabecular and cortical bone parameters. (FIG. 4P) Three-point bending assay. (FIG. 4Q) Serum P1NP. (FIG. 4R) Serum CTX-1. (FIG. 4S) Static histomorphometry. (FIG. 4T) Dynamic histomorphometry (3 month old, male, n=5). Error bars, SD.

FIGS. 5A-M. OX1R Inhibits Osteoblastogenesis by Suppressing Local Ghrelin Expression. (FIGS. 5A-B) Ghrelin protein levels in tibiae (FIG. 5A) and serum (FIG. 5B) of WT or mutant mice (n=2). (FIG. 5C) Ghrelin mRNA levels in tibiae of WT or mutant mice (n=3). (FIGS. 5D-E) Ghrelin mRNA in osteoblast (FIG. 5D) and adipocyte (FIG. 5E) differentiation cultures from the marrow of OX1R-KO or WT mice (n=3). (FIGS. 5F-H) Effect of OX-A, OX-B, OX1R-I, OX2R-I or 1R2R-I (10 nM) on ghrelin expression in marrow differentiation cultures. (FIGS. 5F-G) Ghrelin mRNA in osteoblast (FIG. 5F) and adipocyte (FIG. 5G) cultures (n=3). (FIG. 5H) Ghrelin protein expression and secretion in osteoblast culture (n=2). (FIGS. 5I-M) Ghrelin knockdown abolished the enhanced osteoblastogenesis, reduced adipogenesis, and altered RANKL/OPG expression in OX1R-KO differentiation cultures (n=3). Marrow MSCs were transfected with ghrelin-siRNA (si-Ghrl) or control siRNA (si-Ctrl) (n=3). The results are shown as mRNA expression of ghrelin (FIG. 5I), osteoblast markers (FIG. 5J), adipocyte markers (FIG. 5K), RANKL (FIG. 5L) and OPG (FIG. 5M). “+” and “n.s.” in FIGS. 5D-E compares OX1R-KO with WT control under the same culture conditions; “+” and “n.s.” in FIGS. 5F-G compares treatment with vehicle control under the same culture conditions; in FIGS. 5I-M, “*” compares si-Ghrl with si-Ctrl in OX1R-KO cells, “+” and “n.s.” compares OX1R-KO cells transfected with si-Ghrl or si-Ctrl with WT cells transfected with si-Ctrl. Error bars, SD.

FIGS. 6A-K. OX2R Augments Bone Formation by Suppressing Serum Leptin Level. (FIGS. 6A-C) Leptin protein levels in tibiae (FIG. 6A) and serum (FIG. 6B, FIG. 6C), quantified by western blot (FIGS. 6A-B) (n=2) and ELISA (C) (n=5). (FIG. 6D) Leptin mRNA in WAT (n=3). * in C and D compares mutant mice with WT controls. (FIG. 6E) Serum leptin levels were decreased by ICV injection of OX2R-AG in WT mice (n=5) but not in Ob/Ob mice (n=4). (FIG. 6F) Expression of UCP1, PGC1α and Dio2 in BAT (n=5). (FIG. 6G) NPY expression in hypothalamus (n=5). * and n.s. in FIG. 6F and FIG. 6G compares mutant mice with WT control mice. (FIGS. 6H-L) The bone enhancing effects of OX2R-AG via ICV injection was abolished in Ob/Ob mice (12 week old male, n=4). (FIGS. 6H-K) μCT of tibiae. (FIG. 6H) Images of the trabecular bone of the tibial metaphysis (scale bar, 10 μm). (FIGS. 6I-K) Trabecular bone parameters. (FIG. 6L) Serum P 1NP. Error bars, SD. (FIG. 6M) A simplified working model for how orexin regulates skeletal homeostasis via a ying-yang dual mechanism. On one hand, orexin activation of OX2R in the brain centrally enhances bone mass via a dominant neuroendocrine mechanism by decreasing circulating leptin levels, thus ameliorating the bone-suppressive effects of leptin. On the other hand, orexin activation of OX1R in the bone peripherally reduces bone mass via a subordinate autocrine/paracrine mechanism by decreasing local ghrelin expression, thus compromising the bone-augmenting effects of ghrelin. Consequently, OX1R deletion causes bone gain whereas OX2R deletion causes bone loss; global orexin deletion leads to bone loss whereas global orexin over-expression leads to bone gain.

FIGS. 7A-D. Body weight and gene expression, related to FIG. 1A-L and FIGS. 2A-Z. (FIG. 7A) Body weight was unaltered in the mutant mice compared to WT controls (3 month old, male, chow diet, n=5). (FIG. 7B) Expression of OX-2R was detected in the brain, indicating that the QPCR primers used for the RT-QPCR analysis in FIGS. 2A-B were functional. (FIG. 7C) Orexin expression in the tibiae of mutant and control mice (3 month old, male, chow diet, n=5). (FIG. 7D) TNFα and TRAP expression was detected in macrophage (Mf) and osteoclast (Oc), respectively, indicating that the cDNA for the RT-QPCR analysis in FIG. 2A (right) was intact. Error bars, SD.

FIGS. 8A-B. Expression of osteoblast and adipocyte markers in WT bone marrow differentiation cultures, related to FIGS. 2A-Z. (FIG. 8A) Expression of osteoblast markers in bone marrow osteoblast differentiation cultures (n=3). (FIG. 8B) Expression of adipocyte markers in bone marrow adipocyte differentiation cultures (n=3). Error bars, SD.

FIGS. 9A-F. Additional analyses of orexin regulation of osteoblast and adipocyte differentiation, related to FIGS. 2A-Z. (FIGS. 9A-B) Effects of orexin-A (OX-A, 10 nM) or orexin-B (OX-B, 10 nM) on bone cell differentiation from WT marrow (n=3). (FIG. 9A) Additional osteoblast differentiation markers. (FIG. 9B) Additional adipocyte differentiation marker. (FIGS. 9C-D) Effects of OX1R inhibitor SB-408124 (OX1R-I, 10 nM), OX2R inhibitor compound 1 (OX2R-I, 10 nM) or OX1R2R dual inhibitor ACT-078573/almorexant (1R2R-I, 10 nM) on bone cell differentiation from WT marrow (n=3) Inhibitors were added throughout the differentiation. (FIG. 9C) Additional osteoblast differentiation markers. (FIG. 9D) Additional adipocyte differentiation marker. “+” and “n.s.” in FIGS. 9A-D compares treatment with vehicle control. (FIGS. 9E-F) Effects of anti-orexin-A antibody (α-OX-A, 500 ng/ml) or IgG control on bone cell differentiation from WT marrow (n=3). (FIG. 9E) Osteoblast differentiation markers. (FIG. 9F) Adipocyte differentiation markers. Error bars, SD.

FIGS. 10A-B. Bone formation and leptin level in female OX2R-KO mice, related to FIGS. 3A-Z. Female OX2R-KO mice and littermate WT controls (2 month old, n=5) were compared. (FIG. 10A) Dynamic histomorphometry. (FIG. 10B) ELISA analyses of serum leptin. Error bars, SD.

FIGS. 11A-D. The bone enhancing effects of central administration of an OX2R-AG via ICV injection was abolished in OX2R-KO mice, related to FIGS. 3A-Z. [Ala11, D-Leu15] orexin-B was continuously infused in the lateral ventricles of 10-week-old OX2R-KO male mice at 0.5 nmol/day for 35 days (n=4). (FIGS. 11A-C) Tibiae from OX2R-AG- or vehicle-treated OX2R-KO mice were analyzed by μCT. (FIG. 11A) BV/TV, bone volume/tissue volume ratio. (FIG. 11B) Tb.N, trabecular number. (FIG. 11C) Tb.Sp, trabecular separation. (FIG. 11D) Serum P1NP was unaltered. Error bars, SD.

FIGS. 12A-D. OX1R2R double heterozygous mice do not exhibit significant bone phenotype, related to FIGS. 4A-T. Comparison of 3-month-old male OX1R OX2R double heterozygous mice (1R2R-Het, n=8) with WT controls (n=9). (FIGS. 12A-C) μCT analysis of proximal tibiae. (FIG. 12A) BV/TV, bone volume/tissue volume ratio. (FIG. 12B) Tb.N, trabecular number. (FIG. 12C) Tb.Sp, trabecular separation. (FIG. 12D) Serum P1NP. Error bars, SD.

FIGS. 13A-D. Ghrelin knockdown decreases osteoblast differentiation, related to FIGS. 5A-M. (FIGS. 13A-C) Ghrelin knockdown decreases osteoblast differentiation in WT bone marrow differentiation cultures. WT marrow mesenchymal stem cells were transfected with ghrelin-siRNA (si-Ghrl) or control siRNA (si-Ctrl) (n=3). (FIG. 13A) Ghrelin knockdown reduced ghrelin expression. (FIGS. 13B-C) Ghrelin knockdown decreased the expression of osteoblast differentiation markers Osterix (FIG. 13B) and Collal (FIG. 13C). (FIG. 13D) Ghrelin knockdown reduced ghrelin protein expression in OX1R-KO cultures to a similar level as in WT control cultures. NT, no transfection; G, Ghrelin. Error bars, SD.

### DETAILED DESCRIPTION OF ILLUSTRATIVE EMBODIMENTS

Over 200 million people worldwide suffer from bone disorders such as osteoporosis, bone fractures, and periodontal (gum) disease (where the teeth loose surrounding bone). Osteoporosis represents a large and rapidly growing health care problem with an unmet medical need for therapies that stimulate bone formation. Most current drugs for osteoporosis retard bone degradation but do not stimulate bone formation to replace already lost bone. Compounds that stimulate bone formation thus represent an unmet need in the area of bone disease. Osteoporosis is known to affect approximately 100 million people worldwide—35 million of whom live in the U.S., Western Europe and Japan. Moreover, over 25 million individuals suffer bone fractures yearly, 60 million have periodontal disease (in which the tooth loosens from the jaw bone), and another 18 million have other bone disorders such as bone cancer.

Most current therapies for osteoporosis patients focus on prevention of bone loss, not bone formation. This remains an important consideration as significant morbidity and mortality are associated with prolonged bed rest in the elderly that occur post-bone fracture, particularly those who have suffered hip fractures. Complications of bed rest include blood clots and pneumonia. These complications are recognized and measures are usually taken to avoid them, but these are hardly the best approach to therapy.

Yet another bone-related health issues is bone reconstruction and, specifically, the ability to reconstruct defects in bone tissue that result from traumatic injury, as a consequence of cancer or cancer surgery, as a result of a birth defect, or as a result of aging. There is a significant need for more frequent orthopedic implants, and cranial and facial bone are particular targets for this type of reconstructive need. The availability of new implant materials, e.g., titanium, has permitted the repair of relatively large defects. Titanium implants provide excellent temporary stability across bony defects. However, experience has shown that a lack of viable bone bridging the defect can result in exposure of the appliance, infection, structural instability and, ultimately, failure to repair the defect.

Autologous bone grafts are another possibility to deal with bone injury, but they have several demonstrated disadvantages in that they must be harvested from a donor site such as iliac crest or rib, they usually provide insufficient bone to completely fill the defect, and the bone that does form is sometimes prone to infection and resorption. Partially purified xenogeneic preparations are not practical for clinical use because microgram quantities are purified from kilograms of bovine bone, making large scale commercial production both costly and impractical. Allografts and demineralized bone preparations are therefore often employed. Microsurgical transfers of free bone grafts with attached soft tissue and blood vessels can close bony defects and allow an immediate source of blood supply to the graft. However, these techniques are time consuming, have been shown to produce a great deal of morbidity, and can only be used by specially trained individuals.

Another form of bone disease is that resulting from cancer. A number of cancers metastasize to bone and can result in bone weakening, and some are even associated with bone destruction and bone loss, such as breast, lung, thyroid, kidney and prostate cancer. In addition, Multiple Myeloma and its associated myeloma bone disease (MBD) is not a metastatic cancer. Rather, myeloma cells are derived from the B-cells of the immune system that normally reside in the bone marrow and are therefore intimately associated with bone. Indeed, the bone marrow microenvironment plays an important role in the growth, survival and resistance to chemotherapy of the myeloma cells, which, in turn, regulate the increased bone loss associated with this disorder. Over 90% of myeloma patients have bone involvement, versus 40-60% of cancer patients who have bone metastasis, and over 80% of these MBD patients have intractable bone pain. Additionally, approximately 30% of myeloma patients have hypercalcemia that is a result of the increased osteolytic activity associated with this disease.

Unlike the osteolysis associated with other bone tumors, the MBD lesions are unique in that they do not heal or repair, despite the patients' having many years of complete remission. Mechanistically, this seems to be related to the inhibition and/or loss of the bone-forming osteoblast during disease progression. Indeed, bone marker studies and histomorphometry indicate that both the bone-resorbing osteoclast and osteoblast activity are increased, but balanced early in the disease, whereas overt MBD shows high osteoclast activity and low osteoblast activity. Thus, MBD is a disorder in which bone formation and bone loss are uncoupled and would benefit from therapies that both stimulate bone formation and retard its loss.

To date generally applicable and successful therapies for these types of diseases do not exist. Therefore, there continues to be a need for improved methods of stimulating bone formation and increasing bone strength in vivo to treat bone disease and injury, including cancer. In this study, using both genetic and pharmacological strategies, the inventor uncovered orexin as a “yin-yang” dual regulator of bone mass accrual: on one hand, orexin enhances bone formation via a primary OX2R- and leptin-mediated neuroendocrine control; on the other hand, orexin also suppresses bone formation via a secondary OX1R- and ghrelin-mediated local regulation of bone cell differentiation. These and other aspects of the invention are described in detail below.

### I. BONE STRUCTURE AND PHYSIOLOGY

Bone is a living, growing tissue. It is porous and mineralized, and made up of cells, vessels, organic matrix and inorganic hydroxyapatite crystals. The human skeleton is actually made up of 2 types of bones: the cortical bone and the trabecular bone. Cortical bone represents nearly 80% of the skeletal mass. Cortical bone has a slow turnover rate and a high resistance to bending and torsion. It provides strength where bending would be undesirable as in the middle of long bones. Trabecular bone only represents 20% of the skeletal mass, but 80% of the bone surface. It is less dense, more elastic and has a higher turnover rate than cortical bone.

A. Bone Forming Cells

Osteoprogenitors. Human bone precursor cells are characterized as small-sized cells that express low amounts of bone proteins (osteocalcin, osteonectin, and alkaline phosphatase) and have a low degree of internal complexity (Long et al., 1995). When stimulated to differentiate, these preosteoblast cells become osteoblast in their appearance, size, antigenic expression, and internal structure. Although these cells are normally present at very low frequencies in bone marrow, a process for isolating these cells has been described (Long et al., 1995). U.S. Pat. No. 5,972,703 further describes methods of isolating and using bone precursor cells, and is specifically incorporated herein by reference.

A number of studies indicate that bone marrow derived cells have osteogenic potential. The majority of these investigations point to mesenchymal stem cells (MSC) as undergoing differentiation into osteoblasts when cultured in the presence of bone-active cytokines (Jaiswal et al., 2000; Phinney et al., 1999; Aubin, 1998; Zohar et al., 1997). Mesenchymal stem cells are a pluripotent population capable of generating multiple stromal cell lineages. MSC, as currently used, are a heterogeneous population of cells isolated by plastic adherence, and propagated by low-density passage. Nonetheless, a recent publication indicates the clonal nature of cell fate outcomes in MSC indicating that a single MSC cell can give rise to two or three mesenchymal lineages one of which is usually bone cells (Pittenger et al., 1999). These studies are consistent with earlier reports that demonstrated the osteogenic potential of bone marrow stromal cells, in particular the so-called CFU-f from both mice and human (Friedenstein et al., 1968; Reddi and Huggins, 1972; Friedenstein et al., 1982; Ashton et al., 1985; Bleiberg, 1985; Gronthos et al., 1994; Gronthos et al., 1999).

Single-cell isolation of human MSC generated clones that express the same surface phenotype as unfractionated MSC (Pittenger et al., 1999). Interestingly, of the 6 MSC clones evaluated, 2 retained osteogenic, chrondrogenic and adipogenic potential; others were bipotent (either osteo- plus chondrogenic potential, or osteo-adipocytic potential) or were uni-lineage (chondrocyte). This suggests that MSC themselves are heterogeneous in nature (although culture conditions also may have led to loss of lineage potential). To date, the self-renewal capacity of MSC remains in question. Nonetheless, these in vitro studies and other in vivo studies (Kadiyala et al., 1997; Petite et al., 2000; Krebsbach et al., 1999) show that MSC can commit to the bone cell lineage and develop to the state of matrix mineralization in vitro, or bone formation in vivo.

Preosteoblasts. Preosteoblasts are intermediate between osteoprogenitor cells and osteoblasts. They show increasing expression of bone phenotypic markers such as alkaline phosphatase (Kale et al., 2000). They have a more limited proliferative capacity, but nonetheless continue to divide and produce more preosteoblasts or osteoblasts.

Osteoblasts. An osteoblast is a mononucleate cell that is responsible for bone formation. Osteoblasts produce osteoid, which is composed mainly of Type I collagen. Osteoblasts are also responsible for mineralization of the osteoid matrix. Bone is a dynamic tissue that is constantly being reshaped by osteoblasts, which build bone, and osteoclasts, which resorb bone. Osteoblast cells tend to decrease in number and activity as individuals become elderly, thus decreasing the natural renovation of the bone tissue.

Osteoblasts arise from osteoprogenitor cells located in the periosteum and the bone marrow. Osteoprogenitors are immature progenitor cells that express the master regulatory transcription factor Cbfa1/Runx2. Osteoprogenitors are induced to differentiate under the influence of growth factors, in particular the bone morphogenetic proteins (BMPs). Aside from BMPs, other growth factors including fibroblast growth factor (FGF), platelet-derived growth factor (PDGF), transforming growth factor β (TGF-β) may promote the division of osteoprogenitors and potentially increase osteogenesis. Once osteoprogenitors start to differentiate into osteoblasts, they begin to express a range of genetic markers including Osterix, Col1, ALP, osteocalcin, osteopontin, and osteonectin. Although the term osteoblast implies an immature cell type, osteoblasts are in fact the mature bone cells entirely responsible for generating bone tissue in animals and humans.

Osteoclasts. An osteoclast is a type of bone cell that removes bone tissue by removing its mineralized matrix. This process is known as bone resorption. Osteoclasts and osteoblasts are instrumental in controlling the amount of bone tissue: osteoblasts form bone, osteoclasts resorb bone. Osteoclasts are formed by the fusion of cells of the monocyte-macrophage cell lineage. Osteoclasts are characterized by high expression of tartrate resistant acid phosphatase (TRAP) and cathepsin K.

Osteoclast formation requires the presence of RANK ligand (receptor activator of nuclear factor κβ) and M-CSF (Macrophage colony-stimulating factor). These membrane bound proteins are produced by neighbouring stromal cells and osteoblasts; thus requiring direct contact between these cells and osteoclast precursors. M-CSF acts through its receptor on the osteoclast, c-fms (colony stimulating factor 1 receptor), a transmembrane tyrosine kinase-receptor, leading to secondary messenger activation of tyrosine kinase Src. Both of these molecules are necessary for osteoclastogenesis and are widely involved in the differentiation of monocyte/macrophage derived cells. RANKL is a member of the tumor necrosis family (TNF), and is essential in osteoclastogenesis. RANKL knockout mice exhibit a phenotype of osteopetrosis and defects of tooth eruption, along with an absence or deficiency of osteoclasts. RANKL activates NF-κβ (nuclear factor-κβ) and NFATc1 (nuclear factor of activated t cells, cytoplasmic, calcineurin-dependent 1) through RANK. NF-κβ activation is stimulated almost immediately after RANKL-RANK interaction occurs, and is not upregulated. NFATc1 stimulation, however, begins ˜24-48 hours after binding occurs and its expression has been shown to be RANKL dependent. Osteoclast differentiation is inhibited by osteoprotegerin (OPG), which binds to RANKL thereby preventing interaction with RANK.

B. Bone Formation

The formation of bone during the fetal stage of development occurs by two processes: intramembranous ossification and endochondral ossification. Intramembranous ossification mainly occurs during formation of the flat bones of the skull; the bone is formed from mesenchyme tissue. The steps in intramembranous ossification are development of ossification center, calcification, formation of trabeculae and development of periosteum. Endochondral ossification, on the other hand, occurs in long bones, such as limbs; the bone is formed around a cartilage template. The steps in endochondral ossification are development of cartilage model, growth of cartilage model, development of the primary ossification center and development of the secondary ossification center.

Endochondral ossification begins with points in the cartilage called “primary ossification centers.” They mostly appear during fetal development, though a few short bones begin their primary ossification after birth. They are responsible for the formation of the diaphyses of long bones, short bones and certain parts of irregular bones. Secondary ossification occurs after birth, and forms the epiphyses of long bones and the extremities of irregular and flat bones. The diaphysis and both epiphyses of a long bone are separated by a growing zone of cartilage (the epiphyseal plate). When the child reaches skeletal maturity (18 to 25 years of age), all of the cartilage is replaced by bone, fusing the diaphysis and both epiphyses together (epiphyseal closure).

Remodeling or bone turnover is the process of resorption followed by replacement of bone with little change in shape and occurs throughout a person's life. Osteoblasts and osteoclasts, coupled together via paracrine cell signalling, are referred to as bone remodeling units. The purpose of remodeling is to regulate calcium homeostasis, repair micro-damaged bones (from everyday stress) but also to shape and sculpture the skeleton during growth.

The process of bone resorption by the osteoclasts releases stored calcium into the systemic circulation and is an important process in regulating calcium balance. As bone formation actively fixes circulating calcium in its mineral form, removing it from the bloodstream, resorption actively unfixes it thereby increasing circulating calcium levels. These processes occur in tandem at site-specific locations.

Repeated stress, such as weight-bearing exercise or bone healing, results in the bone thickening at the points of maximum stress (Wolff's law). It has been hypothesized that this is a result of bone's piezoelectric properties, which cause bone to generate small electrical potentials under stress.

### II. OREXIN AND OREXIN RECEPTORS

A. Background

Orexin, also called hypocretin, is a neurotransmitter that regulates arousal, wakefulness, and appetite. The most common form of narcolepsy, in which the sufferer briefly loses muscle tone (cataplexy), is caused by a lack of orexin in the brain due to destruction of the cells that produce it. The brain contains very few cells that produce orexin; in a human brain, about 10,000 to 20,000 neurons in the hypothalamus. However, the axons from these neurons extend throughout the entire brain and spinal cord, where there are also receptors for orexin.

Orexin was discovered almost simultaneously by two independent groups of rat-brain researchers. One group named it orexin, from orexis, meaning “appetite” in Greek; the other group named it hypocretin, because it is produced in the hypothalamus and bears a weak resemblance to secretin, a hormone found in the gut. The scientific community has not yet settled on a consensus for which word to use.

There are two types of orexin: orexin-A and -B (hypocretin-1 and -2). They are excitatory neuropeptide hormones with approximately 50% sequence identity, are produced by cleavage of a single precursor protein. Orexin-A is 33 amino acid residues long and has two intrachain disulfide bonds; orexin-B is a linear 28 amino acid residue peptide. Studies suggest that orexin-A may be of greater biological importance than orexin-B. Although these peptides are produced by a very small population of cells in the lateral and posterior hypothalamus, they send projections throughout the brain. The orexin peptides bind to the two G-protein coupled orexin receptors, OX₁ and OX₂, with orexin-A binding to both OX₁ and OX₂ with approximately equal affinity while orexin-B binds mainly to OX₂ and is 5 times less potent at OX₁. The orexins are strongly conserved peptides, found in all major classes of vertebrates.

B. Orexin Function

The orexin system was initially suggested to be primarily involved in the stimulation of food intake, based on the finding that central administration of orexin-A increases food intake. In addition, it stimulates wakefulness and energy expenditure.

Brown fat activation. Obesity in orexin knockout mice is a result of inability of brown preadipocytes to differentiate into brown adipose tissue (BAT), which in turn reduces BAT thermogenesis. BAT differentiation can be restored in these knockout mice through injections of orexin. Deficiency in orexin has also been linked to narcolepsy, a sleep disorder. Furthermore narcoleptic people are more likely to be obese. Hence obesity in narcoleptic patients may be due to orexin deficiency leading to brown-fat hypo activity and reduced energy expenditure.

Wakefulness. Orexin seems to promote wakefulness. Recent studies indicate that a major role of the orexin system is to integrate metabolic, circadian and sleep debt influences to determine whether an animal should be asleep or awake and active. Orexin neurons strongly excite various brain nuclei with important roles in wakefulness including the dopamine, norepinephrine, histamine and acetylcholine systems and appear to play an important role in stabilizing wakefulness and sleep.

The discovery that an orexin receptor mutation causes the sleep disorder canine narcolepsy in Doberman Pinschers subsequently indicated a major role for this system in sleep regulation. Genetic knockout mice lacking the gene for orexin were also reported to exhibit narcolepsy. Transitioning frequently and rapidly between sleep and wakefulness, these mice display many of the symptoms of narcolepsy. Researchers are using this animal model of narcolepsy to study the disease. Narcolepsy results in excessive daytime sleepiness, inability to consolidate wakefulness in the day (and sleep at night), and cataplexy, which is the loss of muscle tone in response to strong, usually positive, emotions. Dogs that lack a functional receptor for orexin have narcolepsy, while animals and people lacking the orexin neuropeptide itself also have narcolepsy.

Central administration of orexin-A strongly promotes wakefulness, increases body temperature, locomotion and elicits a strong increase in energy expenditure. Sleep deprivation also increases orexin-A transmission. The orexin system may thus be more important in the regulation of energy expenditure than food intake. In fact, orexin-deficient narcoleptic patients have increased obesity rather than decreased BMI, as would be expected if orexin were primarily an appetite stimulating peptide. Another indication that deficits of orexin cause narcolepsy is that depriving monkeys of sleep for 30-36 hours and then injecting them with the neurochemical alleviates the cognitive deficiencies normally seen with such amount of sleep loss.

In humans, narcolepsy is associated with a specific variant of the human leukocyte antigen (HLA) complex. Furthermore, genome-wide analysis shows that, in addition to the HLA variant, narcoleptic humans also exhibit a specific genetic mutation in the T-cell receptor alpha locus. In conjunction, these genetic anomalies cause the immune system to attack and kill the critical orexin neurons. Hence the absence of orexin-producing neurons in narcoleptic humans may be the result of an autoimmune disorder.

Food Intake. Orexin increases the craving for food, and correlates with the function of the substances that promote its production. Leptin is a hormone produced by fat cells and acts as a long-term internal measure of energy state. Ghrelin is a short-term factor secreted by the stomach just before an expected meal, and strongly promotes food intake.

Orexin-producing cells have recently been shown to be inhibited by leptin (through the leptin receptor pathway), but are activated by ghrelin and hypoglycemia (glucose inhibits orexin production). Orexin, as of 2007, is claimed to be a very important link between metabolism and sleep regulation. Such a relationship has been long suspected, based on the observation that long-term sleep deprivation in rodents dramatically increases food intake and energy metabolism, i.e., catabolism, with lethal consequences on a long-term basis.

Pharmacologic potential. The research on orexin mimics is still in an early phase, although many scientists believe that orexin-based drugs could help narcoleptics and increase alertness in the brain without the side effects of amphetamines. Merck reported at the Sleep 2012 conference that insomniacs taking an orexin blocker, suvorexant, fell asleep faster and slept an hour longer. Suvorexant was tested for three months on over a thousand patients in a phase III trial. Preliminary research has been conducted that shows potential for orexin blockers in the treatment of alcoholism. Lab rats given drugs which targeted the orexin system lost interest in alcohol despite being given free access in experiments. A study has reported that transplantation of orexin neurons into the pontine reticular formation in rats is feasible, indicating the development of alternative therapeutic strategies in addition to pharmacological interventions to treat narcolepsy.

Because orexin-A receptors have been shown to regulate relapse to cocaine seeking, a new study investigated its relation to nicotine by studying rats. By blocking the orexin-A receptor with low doses of the selective antagonist SB-334,867, nicotine self-administration decreased and also the motivation to seek and obtain the drug. The study showed that blocking of receptors in the insula decreased self-administration, but not blocking of receptors in the adjacent somatosensory cortex. The greatest decrease in self-administration was found when blocking all orexin-A receptors in the brain as a whole. A rationale for this study was the fact that the insula has been implicated in regulating feelings of craving. The insula contains orexin-A receptors. It has been reported that smokers who sustained damage to the insula lost the desire to smoke.

Lipid metabolism. Orexin-A (OXA) has been recently demonstrated to have direct effect on a part of the lipid metabolism. OXA stimulates glucose uptake in 3T3-L1 adipocytes and that increased energy uptake is stored as lipids (triacylglycerol). OXA thus increases lipogenesis. It also inhibits lipolysis and stimulates the secretion of adiponectin. These effects are thought to be mostly conferred via the PI3K pathway because this pathway inhibitor (LY294002) completely blocks OXA effects in adipocytes. The link between OXA and the lipid metabolism is new and currently under more research. Obesity in orexin-knockout mice is associated with impaired brown adipose tissue thermogenesis.

Mood. High levels of orexin-A have been associated with happiness in human subjects, while low levels have been associated with sadness. The finding suggests that boosting levels of orexin-A could elevate mood in humans, being thus a possible future treatment for disorders like depression. Likewise, it helps explain the incidence of depression associated with narcolepsy.

C. Orexin Receptors

The orexin receptor (also referred to as the hypocretin receptor) is a G-protein-coupled receptor that binds the neuropeptide hormone orexin. There are two variants, OX₁ and OX₂, each encoded by a different gene (HCRTR1, HCRTR2). Both orexin receptors exhibit a similar pharmacology—the 2 orexin peptides, orexin-A and orexin-B, bind to both receptors and, in each case, agonist binding results in an increase in intracellular calcium levels. However, orexin-B shows a 10-fold selectivity for orexin receptor type 2, whilst orexin-A is equipotent at both receptors.

D. Agonists and Antagonists of Orexin Receptors

Several orexin receptor antagonists are in development for potential use in sleep disorders. Several drugs acting on the orexin system are under development, either orexin agonists for the treatment of conditions such as narcolepsy, or orexin antagonists for insomnia. No non-peptide agonists are yet available, although synthetic Orexin-A polypeptide has been made available as a nasal spray and tested on monkeys. Several non-peptide antagonists are in development however; SB-649,868 is under development by GlaxoSmithKline for sleep disorders and is a non-selective orexin receptor antagonist. Another OX₁ and OX₂ receptor antagonist (ACT-078573, almorexant) is a similar compound under development for primary insomnia by Actelion. A third entry is Merck's MK-4305.

Most ligands acting on the orexin system so far are polypeptides modified from the endogenous agonists Orexin-A and Orexin-B. Given the constrasting roles of OX₁ and OX₂ receptors in bone biology, the use of selective modulators is desired for the purposes of this disclosure. There there are some OX₁-selective non-peptide antagonists available, however:
SB-334,867—selective OX₁ antagonist
SB-408,124—selective OX₁ antagonist
   U.S. Patent Publication 20100150840 and PCT WO 03/105779 disclose selective OX₂ agonists. Orexin-B and its variant Ala(11)/d-Leu(15) are selective for OX₂ as well.

### III. METHODS OF TREATMENT

A. Pharmacological Therapeutic Agents and Administration

The present invention addresses therapies, e.g., treatment of bone loss conditions. In various embodiments, the inhibitory agents of the present invention are formulated for administration in pharmacologically acceptable vehicles, such as parenteral, topical, aerosal, liposomal, nasal or ophthalmic preparations. In certain embodiments, formulations may be designed for oral or topical administration. It is further envisioned that formulations of agents that might be delivered may be formulated and administered in a manner that does not require that they be in a single pharmaceutically acceptable carrier. In those situations, it would be clear to one of ordinary skill in the art the types of diluents that would be proper for the proposed use of the polypeptides and any secondary agents required.

The phrases “pharmaceutically” or “pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the compositions, vectors or cells of the present invention, its use in therapeutic compositions is contemplated. Supplementary active ingredients also can be incorporated into the compositions.

The active compositions of the present invention may include classic pharmaceutical preparations. Administration of these compositions according to the present invention will be via any common route so long as the target tissue or surface is available via that route. This includes oral, nasal, or topical. Alternatively, administration may be by introcular, intra-hepatic, orthotopic, intradermal, subcutaneous, intramuscular, intraperitoneal or intravenous injection. Such compositions would normally be administered as pharmaceutically acceptable compositions, described supra.

The active compounds may also be administered parenterally or intraperitoneally. Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The compositions of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

B. Devices

In addition to providing agents for administration by routes discussed above, such agents, alone or in combination, maybe used in the context of devices, such as implants. A variety of bone related implants are contemplated, including dental implants, joint implants such as hips, knees, and elbows, vertebral/spinal implants, and others. The agents may be impregnated in a surface of the implant, including in a bioactive matrix or coating. The agent may be further formulated to sustained, delayed, prolonged or time release. The coating may comprise polymers, for example, such as those listed below. The following is a list of U.S. patents relating to bone implants and devices which may be utilized in accordance with this embodiment of the invention:

TABLE 1
BONE IMPLANT PATENTS

| U.S. Patent* | Patent Title |
|---|---|
| 7,044,972 | Bone implant, in particular, an inter-vertebral |
| | implant |
| 7,022,137 | Bone hemi-lumbar interbody spinal fusion implant |
| | having an asymmetrical leading end and method of |
| | installation thereof |
| 7,001,551 | Method of forming a composite bone material implant |
| 6,994,726 | Dual function prosthetic bone implant and method for |
| | preparing the same |
| 6,989,031 | Hemi-interbody spinal implant manufactured from a |
| | major long bone ring or a bone composite |
| 6,988,015 | Bone implant |
| 6,981,975 | Method for inserting a spinal fusion implant having |
| | deployable bone engaging projections |
| 6,981,872 | Bone implant method of implanting, and kit for use |
| | in making implants, particularly useful with respect |
| | to dental implants |
| 6,929,662 | End member for a bone fusion implant |
| 6,923,830 | Spinal fusion implant having deployable bone |
| | engaging projections |
| 6,921,264 | Implant to be implanted in bone tissue or in bone |
| | tissue supplemented with bone substitute material |
| 6,918,766 | Method, arrangement and use of an implant for |
| | ensuring delivery of bioactive substance to the bone |
| | and/or tissue surrounding the implant |
| 6,913,621 | Flexible implant using partially demineralized bone |
| 6,899,734 | Modular implant for fusing adjacent bone structure |
| 6,860,884 | Implant for bone connector |
| 6,852,129 | Adjustable bone fusion implant and method |
| 6,802,845 | Implant for bone connector |
| 6,786,908 | Bone fracture support implant with non-metal spacers |
| 6,767,367 | Spinal fusion implant having deployable bone |
| | engaging projections |
| 6,761,738 | Reinforced molded implant formed of cortical bone |
| 6,755,832 | Bone plate implant |
| 6,730,129 | Implant for application in bone, method for |
| | producing such an implant, and use of such an |
| | implant |
| 6,689,167 | Method of using spinal fusion device, bone joining |
| | implant, and vertebral fusion implant |
| 6,689,136 | Implant for fixing two bone fragments to each other |
| 6,666,890 | Bone hemi-lumbar interbody spinal implant having an |
| | asymmetrical leading end and method of installation |
| | thereof |
| 6,652,592 | Segmentally demineralized bone implant |
| 6,648,917 | Adjustable bone fusion implant and method |
| 6,607,557 | Artificial bone graft implant |
| 6,599,322 | Method for producing undercut micro recesses in a |
| | surface, a surgical implant made thereby, and method |
| | for fixing an implant to bone |
| 6,562,074 | Adjustable bone fusion implant and method |
| 6,562,073 | Spinal bone implant |
| 6,540,770 | Reversible fixation device for securing an implant |
| | in bone |
| 6,537,277 | Implant for fixing a bone plate |
| 6,506,051 | Bone implant with intermediate member and expanding |
| | assembly |
| 6,478,825 | Implant, method of making same and use of the |
| | implant for the treatment of bone defects |
| 6,458,136 | Orthopaedic instrument for sizing implant sites and |
| | for pressurizing bone cement and a method for using |
| | the same |
| 6,447,545 | Self-aligning bone implant |
| 6,436,146 | Implant for treating ailments of a joint or a bone |
| 6,371,986 | Spinal fusion device, bone joining implant, and |
| | vertebral fusion implant |
| 6,370,418 | Device and method for measuring the position of a |
| | bone implant |
| 6,364,880 | Spinal implant with bone screws |
| 6,350,283 | Bone hemi-lumbar interbody spinal implant having an |
| | asymmetrical leading end and method of installation |
| | thereof |
| 6,350,126 | Bone implant |
| 6,287,343 | Threaded spinal implant with bone ingrowth openings |
| 6,270,346 | Dental implant for bone regrowth |
| 6,248,109 | Implant for interconnecting two bone fragments |
| 6,217,617 | Bone implant and method of securing |
| 6,214,050 | Expandable implant for inter-bone stabilization and |
| | adapted to extrude osteogenic material, and a method |
| | of stabilizing bones while extruding osteogenic |
| | material |
| 6,213,775 | Method of fastening an implant to a bone and an |
| | implant therefor |
| 6,206,923 | Flexible implant using partially demineralized bone |
| 6,203,545 | Implant for fixing bone fragments after an osteotomy |
| 6,149,689 | Implant as bone replacement |
| 6,149,688 | Artificial bone graft implant |
| 6,149,686 | Threaded spinal implant with bone ingrowth openings |
| 6,126,662 | Bone implant |
| 6,083,264 | Implant material for replacing or augmenting living |
| | bone tissue involving thermoplastic syntactic foam |
| 6,058,590 | Apparatus and methods for embedding a biocompatible |
| | material in a polymer bone implant |
| 6,018,094 | Implant and insert assembly for bone and uses |
| | thereof |
| 5,976,147 | Modular instrumentation for bone preparation and |
| | implant trial reduction of orthopedic implants |
| 5,906,488 | Releasable holding device preventing undesirable |
| | rotation during tightening of a screw connection in |
| | a bone anchored implant |
| 5,899,939 | Bone-derived implant for load-supporting |
| | applications |
| 5,895,425 | Bone implant |
| 5,890,902 | Implant bone locking mechanism and artificial |
| | periodontal ligament system |
| 5,885,287 | Self-tapping interbody bone implant |
| 5,819,748 | Implant for use in bone surgery |
| 5,810,589 | Dental implant abutment combination that reduces |
| | crestal bone stress |
| 5,759,035 | Bone fusion dental implant with hybrid anchor |
| 5,720,750 | Device for the preparation of a tubular bone for the |
| | insertion of an implant shaft |
| 5,709,683 | Interbody bone implant having conjoining |
| | stabilization features for bony fusion |
| 5,709,547 | Dental implant for anchorage in cortical bone |
| 5,674,725 | Implant materials having a phosphatase and an |
| | organophosphorus compound for in vivo mineralization |
| | of bone |
| 5,658,338 | Prosthetic modular bone fixation mantle and implant |
| | system |
| D381,080 | Combined metallic skull base surgical implant and |
| | bone flap fixation plate |
| 5,639,402 | Method for fabricating artificial bone implant green |
| | parts |
| 5,624,462 | Bone implant and method of securing |
| D378,314 | Bone spinal implant |
| 5,607,430 | Bone stabilization implant having a bone plate |
| | portion with integral cable clamping means |
| 5,571,185 | Process for the production of a bone implant and a |
| | bone implant produced thereby |
| 5,456,723 | Metallic implant anchorable to bone tissue for |
| | replacing a broken or diseased bone |
| 5,441,538 | Bone implant and method of securing |
| 5,405,388 | Bone biopsy implant |
| 5,397,358 | Bone implant |
| 5,383,935 | Prosthetic implant with self-generated current for |
| | early fixation in skeletal bone |
| 5,364,268 | Method for installing a dental implant fixture in |
| | cortical bone |
| 5,312,256 | Dental implant for vertical penetration, adapted to |
| | different degrees of hardness of the bone |

*The preceding patents are all hereby incorporated by reference in their entirety.

C. Combined Therapy

In another embodiment, it is envisioned to use the agonists/antagonists of the present invention in combination with other therapeutic modalities. Thus, in addition to the therapies described above, one may also provide to the patient more “standard” pharmaceutical therapies. Combinations may be achieved by contacting cells, tissues or subjects with a single composition or pharmacological formulation that includes both agents, or by contacting the cell with two distinct compositions or formulations, at the same time, wherein one composition includes the agonist/antagonist and the other includes the other agent. Alternatively, the therapy using an agonist/antagonist may precede or follow administration of the other agent(s) by intervals ranging from minutes to weeks. In embodiments where the other agent and agonist/antagonist are applied separately to the cell, one would generally ensure that a significant period of time did not expire between the time of each delivery, such that the agent and the agonist/antagonist would still be able to exert an advantageously combined effect on the cell, tissue or subject. In such instances, it is contemplated that one would typically contact the cell with both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either an agonist/antagonist, or the other agent will be desired. In this regard, various combinations may be employed. By way of illustration, where the agonist(s) and/or antagonist(s) is “A” and the other agent is “B,” the following permutations based on 3 and 4 total administrations are exemplary:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Other combinations are likewise contemplated. Administration protocols and formulation of such agents will generally follow those of standard pharmaceutical drugs, as discussed further below. Combination agents include bisphosphonates (Didronel™, Fosamax™ and Actonel™), SERMs (Evista) or other hormone derivatives, and Parathyroid Hormone (PTH) analogs.

D. Disease States

A plethora of conditions are characterized by the need to enhance bone formation or to inhibit bone resorption and thus would benefit from treatment described herein in promoting bone formation and/or bone repair. Perhaps the most obvious is the case of bone fractures, where it would be desirable to stimulate bone growth and to hasten and complete bone repair. Agents that enhance bone formation would also be useful in facial reconstruction procedures. Other bone deficit conditions include bone segmental defects, periodontal disease, metastatic bone disease, osteolytic bone disease and conditions where connective tissue repair would be beneficial, such as healing or regeneration of cartilage defects or injury. Also of great significance is the chronic condition of osteoporosis, including age-related osteoporosis and osteoporosis associated with post-menopausal hormone status. Other conditions characterized by the need for bone growth include primary and secondary hyperparathyroidism, disuse osteoporosis, diabetes-related osteoporosis, and glucocorticoid-related osteoporosis. Several other conditions, such as, for example, vitamin D deficiency, exists. Finally, bone attacking cacners are another significant application of this technology.

Fracture. The first example is the otherwise healthy individual who suffers a fracture. Often, clinical bone fracture is treated by casting to alleviate pain and allow natural repair mechanisms to repair the wound. There has been progress in the treatment of fracture in recent times, however, even without considering the various complications that may arise in treating fractured bones, any new procedures to increase bone healing in normal circumstances would represent a great advance.

Periodontal Disease. Progressive periodontal disease leads to tooth loss through destruction of the tooth's attachment to the surrounding bone. Approximately 5-20% of the U.S. population (15-60 million individuals) suffers from severe generalized periodontal disease, and there are 2 million related surgical procedures. Moreover, if the disease is defined as the identification of at least one site of clinical attachment loss, then approximately 80% of all adults are affected, and 90% of those aged 55 to 64 years. If untreated, approximately 88% of affected individuals show moderate to rapid progression of the disease which shows a strong correlation with age. The major current treatment for periodontal disease is regenerative therapy consisting of replacement of lost periodontal tissues. The lost bone is usually treated with an individual's own bone and bone marrow, due to their high osteogenic potential. Bone allografts (between individuals) can also be performed using stored human bone. Although current periodontal cost analyses are hard to obtain, the size of the affected population and the current use of bone grafts as a first-order therapy strongly suggest that this area represents an attractive target for bone-building therapies.

Osteopenia/osteoporosis. The terms osteopenia and osteoporosis refers to a heterogeneous group of disorders characterized by decreased bone mass and fractures. Osteopenia is a bone mass that is one or more standard deviations below the mean bone mass for a population; osteoporosis is defined as 2.5 SD or lower. An estimated 20-25 million people are at increased risk for fracture because of site-specific bone loss. Risk factors for osteoporosis include increasing age, gender (more females), low bone mass, early menopause, race (Caucasians in general; asian and hispanic females), low calcium intake, reduced physical activity, genetic factors, environmental factors (including cigarette smoking and abuse of alcohol or caffeine), and deficiencies in neuromuscular control that create a propensity to fall. More than a million fractures in the U.S. each year can be attributed to osteoporosis. In economic terms, the costs (exclusive of lost wages) for osteoporosis therapies are $35 billion worldwide. Demographic trends (i.e., the gradually increasing age of the U.S. population) suggest that these costs may increase to $62 billion by the year 2020. Clearly, osteoporosis is a significant health care problem.

Osteoporosis, once thought to be a natural part of aging among women, is no longer considered age or gender-dependent. Osteoporosis is defined as a skeletal disorder characterized by compromised bone strength predisposing to an increased risk of fracture. Bone strength reflects the integration of two main features: bone density and bone quality. Bone density is expressed as grams of mineral per area or volume and in any given individual is determined by peak bone mass and amount of bone loss. Bone quality refers to architecture, turnover, damage accumulation (e.g., microfractures) and mineralization. A fracture occurs when a failure-inducing force (e.g., trauma) is applied to osteoporotic bone.

Current therapies for osteoporosis patients focus on fracture prevention, not for promoting bone formation or fracture repair. This remains an important consideration because of the literature, which clearly states that significant morbidity and mortality are associated with prolonged bed rest in the elderly, particularly those who have suffered hip fractures. Complications of bed rest include blood clots and pneumonia. These complications are recognized and measures are usually taken to avoid them, but these is hardly the best approach to therapy. Thus, the osteoporotic patient population would benefit from new therapies designed to strengthen bone and speed up the fracture repair process, thus getting these people on their feet before the complications arise.

Bone Reconstruction/Grafting. A fourth example is related to bone reconstruction and, specifically, the ability to reconstruct defects in bone tissue that result from traumatic injury; as a consequence of cancer or cancer surgery; as a result of a birth defect; or as a result of aging. There is a significant need for more frequent orthopedic implants, and cranial and facial bone are particular targets for this type of reconstructive need. The availability of new implant materials, e.g., titanium, has permitted the repair of relatively large defects. Titanium implants provide excellent temporary stability across bony defects and are an excellent material for bone implants or artificial joints such as hip, knee and joint replacements. However, experience has shown that a lack of viable bone binding to implants the defect can result in exposure of the appliance to infection, structural instability and, ultimately, failure to repair the defect. Thus, a therapeutic agent that stimulates bone formation on or around the implant will facilitate more rapid recovery. Autologous bone grafts are another possibility, but they have several demonstrated disadvantages in that they must be harvested from a donor site such as iliac crest or rib, they usually provide insufficient bone to completely fill the defect, and the bone that does form is sometimes prone to infection and resorption. Partially purified xenogeneic preparations are not practical for clinical use because microgram quantities are purified from kilograms of bovine bone, making large scale commercial production both costly and impractical. Allografts and demineralized bone preparations are therefore often employed, but suffer from their devitalized nature in that they only function as scaffolds for endogenous bone cell growth.

Microsurgical transfers of free bone grafts with attached soft tissue and blood vessels can close bony defects with an immediate source of blood supply to the graft. However, these techniques are time consuming, have been shown to produce a great deal of morbidity, and can only be used by specially trained individuals. Furthermore, the bone implant is often limited in quantity and is not readily contoured. In the mandible, for example, the majority of patients cannot wear dental appliances using presently accepted techniques (even after continuity is established), and thus gain little improvement in the ability to masticate.

In connection with bone reconstruction, specific problem areas for improvement are those concerned with treating large defects, such as created by trauma, birth defects, or particularly, following tumor resection; and also the area of artificial joints. The success of orthopaedic implants, interfaces and artificial joints could conceivably be improved if the surface of the implant, or a functional part of an implant, were to be coated with a bone stimulatory agent. The surface of implants could be coated with one or more appropriate materials in order to promote a more effective interaction with the biological site surrounding the implant and, ideally, to promote tissue repair.

Primary Bone Cancer and Metastatic Bone Disease. Bone cancer occurs infrequently while bone metastases are present in a wide range of cancers, including thyroid, kidney, and lung. Metastatic bone cancer is a chronic condition; survival from the time of diagnosis is variable depending on tumor type. In prostate and breast cancer and in multiple myeloma, survival time is measurable in years. For advanced lung cancer, it is measured in months. Cancer symptoms include pain, hypercalcemia, pathologic fracture, and spinal cord or nerve compression. Prognosis of metastatic bone cancer is influenced by primary tumor site, presence of extra-osseous disease, and the extent and tempo of the bone disease. Bone cancer/metastasis progression is determined by imaging tests and measurement of bone specific markers. Recent investigations show a strong correlation between the rate of bone resorption and clinical outcome, both in terms of disease progression or death.

Multiple Myeloma. Multiple myeloma (MM) is a B-lymphocyte malignancy characterized by the accumulation of malignant clonal plasma cells in the bone marrow. The clinical manifestations of the disease are due to the replacement of normal bone marrow components by abnormal plasma cells, with subsequent overproduction of a monoclonal immunoglobulin (M protein or M component), bone destruction, bone pain, anemia, hypercalcemia and renal dysfunction.

As distinct from other cancers that spread to the bone (e.g., breast, lung, thyroid, kidney, prostate), myeloma bone disease (MBD) is not a metastatic disease. Rather, myeloma cells are derived from the B-cells of the immune system that normally reside in the bone marrow and are therefore intimately associated with bone. Indeed, the bone marrow microenvironment plays an important role in the growth, survival and resistance to chemotherapy of the myeloma cells, which, in turn, regulate the increased bone loss associated with this disorder. Over 90% of myeloma patients have bone involvement, versus 40-60% of cancer patients who have bone metastasis, and over 80% have intractable bone pain. Additionally, approximately 30% of myeloma patients have hypercalcemia that is a result of the increased osteolytic activity associated with this disease.

Common problems in myeloma are weakness, confusion and fatigue due to hypercalcemia. Headache, visual changes and retinopathy may be the result of hyperviscosity of the blood depending on the properties of the paraprotein. Finally, there may be radicular pain, loss of bowel or bladder control (due to involvement of spinal cord leading to cord compression) or carpal tunnel syndrome and other neuropathies (due to infiltration of peripheral nerves by amyloid). It may give rise to paraplegia in late presenting cases.

Myeloma Bone Disease. As discussed above, unlike the osteolysis associated with other bone tumors, the MBD lesions are unique in that they do not heal or repair, despite the patients' having many years of complete remission. Mechanistically, this seems to be related to the inhibition and/or loss of the bone-forming osteoblast during disease progression. Indeed, bone marker studies and histomorphometry indicate that both the bone-resorbing osteoclast and osteoblast activity are increased, but balanced early in the disease, whereas overt MBD shows high osteoclast activity and low osteoblast activity. Thus, MBD is a disorder in which bone formation and bone loss are uncoupled and would benefit from therapies that both stimulate bone formation and retard its loss.

A number of therapeutic approaches have been used in MBD, with the endpoints of treating pain, hypercalcemia, or the reduction of skeletal related events (SRE). Many of these may present serious complications. Surgery, such as vertebroplasty or kyphoplasty, that is performed for stability and pain relief has the attendant surgical risks (e.g., infection) made worse by a compromised immune system and does not reverse existing skeletal defects. Radiation therapy and radioisotope therapy are both used to prevent/control disease progression and have the typical risks of irradiation therapies. More recently, drugs such as the bisphosphonates that inhibit osteoclast activity have become a standard of therapy for MBD, despite the fact that they work poorly in this disorder. In 9 major double-blind, placebo-controlled trials on bisphosphonates, only 66% of patients showed an effective reduction in pain; 56% showed a reduction in SRE and only 1 of the 9 demonstrated a survival benefit.

Rheumatoid Arthritis. Rheumatoid arthritis (RA) is an autoimmune disease that results in a chronic, systemic inflammatory disorder that may affect many tissues and organs, but principally attacks flexible (synovial) joints. It can be a disabling and painful condition, which can lead to substantial loss of functioning and mobility if not adequately treated. The process involves an inflammatory response of the capsule around the joints (synovium) secondary to swelling (turgescence) of synovial cells, excess synovial fluid, and the development of fibrous tissue (pannus) in the synovium. The pathology of the disease process often leads to the destruction of articular cartilage and ankylosis (fusion) of the joints. RA can also produce diffuse inflammation in the lungs, the membrane around the heart (pericardium), the membranes of the lung (pleura), and white of the eye (sclera), and also nodular lesions, most common in subcutaneous tissue. Although the cause of RA is unknown, autoimmunity plays a big part, and RA is a systemic autoimmune disease. It is a clinical diagnosis made on the basis of symptoms, physical exam, radiographs (X-rays) and labs.

Treatments are pharmacological and non-pharmacological. Non-pharmacological treatment includes physical therapy, orthoses, occupational therapy and nutritional therapy but these don't stop the progression of joint destruction. Analgesia (painkillers) and anti-inflammatory drugs, including steroids, suppress symptoms, but don't stop the progression of joint destruction either. Disease-modifying antirheumatic drugs (DMARDs) slow or halt the progress of the disease. The newer biologics are DMARDs. The evidence for complementary and alternative medicine (CAM) treatments for RA related pain is weak, with the lack of high quality evidence leading to the conclusions that their use is currently not supported by the evidence nor proved to be of benefit.

About 0.6% of the United States adult population has RA, women two to three times as often as men. Onset is most frequent during middle age, but people of any age can be affected. RA primarily affects joints, however it also affects other organs in 15-25% of individuals. It can be difficult to determine whether disease manifestations are directly caused by the rheumatoid process itself, or from side effects of the medications used to treat it—for example, lung fibrosis from methotrexate or osteoporosis from corticosteroids.

Arthritis of joints involves inflammation of the synovial membrane. Joints become swollen, tender and warm, and stiffness limits their movement. With time multiple joints are affected (it is a polyarthritis). Most commonly involved are the small joints of the hands, feet and cervical spine, but larger joints like the shoulder and knee can also be involved. Synovitis can lead to tethering of tissue with loss of movement and erosion of the joint surface causing deformity and loss of function.

RA typically manifests with signs of inflammation, with the affected joints being swollen, warm, painful and stiff, particularly early in the morning on waking or following prolonged inactivity. Increased stiffness early in the morning is often a prominent feature of the disease and typically lasts for more than an hour. Gentle movements may relieve symptoms in early stages of the disease. These signs help distinguish rheumatoid from non-inflammatory problems of the joints, often referred to as osteoarthritis or “wear-and-tear” arthritis. In arthritis of non-inflammatory causes, signs of inflammation and early morning stiffness are less prominent with stiffness typically less than 1 hour, and movements induce pain caused by mechanical arthritis. In RA, the joints are often affected in a fairly symmetrical fashion, although this is not specific, and the initial presentation may be asymmetrical.

Local osteoporosis occurs in RA around inflamed joints. It is postulated to be partially caused by inflammatory cytokines. More general osteoporosis is probably contributed to by immobility, systemic cytokine effects, local cytokine release in bone marrow and corticosteroid therapy.

Paget's Disease. Paget's disease of bone is a chronic disorder that can result in enlarged and misshapen bones. The excessive breakdown and formation of bone tissue causes affected bone to weaken, resulting in pain, misshapen bones, fractures, and arthritis in the joints near the affected bones. Paget's disease typically is localized, affecting just one or a few bones, as opposed to osteoporosis, for example, which usually affects all the bones in the body. Decisions about treating Paget's disease can be complicated because no two people are affected in exactly the same way by the disease, and because it is sometimes difficult to predict whether a person with Paget's disease who shows no signs of the disorder will develop symptoms or complications (such as a bone fracture) at a later date. Although there is no cure for Paget's disease, medications can help control the disorder and lessen pain and other symptoms. Paget's disease experts recommend that these medications be taken by people with Paget's disease who have bone pain, headache, back pain, or a nerve-related symptom (such as “shooting” pains in the leg) that is directly associated with the disease; have elevated levels of serum alkaline phosphatase (ALP) in their blood; display evidence that a bone fracture will occur; require pretreatment therapy for affected bones that require surgery; have active symptoms in the skull, long bones, or vertebrae (spine); have the disease in bones located next to major joints, placing them at risk of developing osteoarthritis; develop hypercalcemia that occurs when a person with several bones affected by Paget's disease and a high serum alkaline phosphatase level is immobilized.

Today's medications, especially when started before complications begin, are often successful in controlling the disorder. Paget's disease is rarely diagnosed in people less than 40 years of age. Men are more commonly affected than women (3:2). Prevalence of Paget's disease ranges from 1.5 to 8.0 percent, depending on age and country of residence. Prevalence of familial Paget's disease (where more than one family member has the disease) ranges from 10 to 40 percent in different parts of the world. Because early diagnosis and treatment is important, after age 40, siblings and children of someone with Paget's disease may wish to have an alkaline phosphatase blood test every two or three years. If the alkaline phosphatase level is above normal, other tests such as a bone-specific alkaline phosphatase test, bone scan, or X-ray can be performed.

The pathogenesis of Paget's disease is described in 4 stages: osteoclastic activity; mixed osteoclastic-osteoblastic activity; osteoblastic activity; and malignant degeneration. Initially, there is a marked increase in the rate of bone resorption at localized areas caused by large and numerous osteoclasts. These localized areas of osteolysis are seen radiologically as an advancing lytic wedge in long bones or osteoporosis circumscripta in the skull. The osteolysis is followed by a compensatory increase in bone formation induced by osteoblasts recruited to the area. This is associated with accelerated deposition of lamellar bone in a disorganized fashion. This intense cellular activity produces a chaotic picture of trabecular bone (“mosaic” pattern), rather than the normal linear lamellar pattern. The resorbed bone is replaced and the marrow spaces are filled by an excess of fibrous connective tissue with a marked increase in blood vessels, causing the bone to become hypervascular. The bone hypercellularity may then diminish, leaving a dense “pagetic bone,” also known as burned-out Paget's disease.

The goal of treatment is to relieve bone pain and prevent the progression of the disease. Five bisphosphonates are currently available. In general, the most commonly prescribed are risedronic acid (Actonel), alendronic acid (Fosamax), and pamidronic acid (Aredia). Etidronic acid (Didronel) and other bisphosphonates may be appropriate therapies for selected patients but are less commonly used. As a rule, bisphosphonate tablets should be taken with 200-250 mL (6-8& oz) of tap water (not from a source with high mineral content) on an empty stomach. None of these drugs should be used by people with severe kidney disease.

Etidronate disodium (Didronel) in tablet form is available in 200-400 mg doses. The approved regimen is once daily for six months; the higher dose (400 mg) is more commonly used. No food, beverage, or medications should be consumed for two hours before and after taking. The course should not exceed six months, but repeat courses can be given after rest periods, preferably of three to six months duration.

Pamidronate disodium (Aredia) in intravenous form: the approved regimen uses a 30 mg infusion over four hours on each of three consecutive days, but a more commonly used regimen is 60 mg over two to four hours for two or more consecutive or nonconsecutive days.

Alendronate sodium (Fosamax) is given as tablets of 40 mg once daily for six months; patients should wait at least 30 minutes after taking before eating any food, drinking anything other than tap water, taking any medication, or lying down (patient may sit).

Tiludronate disodium (Skelid) in two tablets of 200 mg are taken once daily for three months; they may be taken any time of day, as long as there is a period of two hours before and after resuming food, beverages, and medications.

Risedronate sodium (Actonel) as a 30 mg tablet taken once daily for 2 months is the prescribed regimen; patients should wait at least 30 minutes after taking before eating any food, drinking anything other than tap water, taking any medication, or lying down (patient may sit).

Zoledronic acid (Reclast, Aclasta) is given as an intravenous infusion; a single dose (5 mg over 15 minutes) is effective for two years.

Miacalcin is administered by injection; 50 to 100 units daily or three times per week for 6-18 months. Repeat courses can be given after brief rest periods. Miacalcin may be appropriate for certain patients, but is seldom used. However, it is to be remembered that calcitonin is also linked to increased chance of cancer. The European equivalent of the US Food and Drug Administration (FDA) recommended withdrawing calcitonin nasal spray because of an increased risk for cancer.

Risk. The present invention also contemplates treating individuals at risk for any of the aforementioned disease states. These individuals would include those persons suffering from conditions discussed above.

### IV. KITS

Any of the compositions described herein may be comprised in a kit. In a non-limiting example, an individual agonist or antagonist is included in a kit. The kit may also include one or more transfection reagent(s) to facilitate delivery of the agonist to cells.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there is more than one component in the kit (labeling reagent and label may be packaged together), the kit also will generally contain a second, third or other additional container into which the additional components may be separately placed. However, various combinations of components may be comprised in a vial. The kits of the present invention also will typically include a means for containing the agents, and any other reagent containers in close confinement for commercial sale. Such containers may include injection or blow-molded plastic containers into which the desired vials are retained.

When the components of the kit are provided in one and/or more liquid solutions, the liquid solution is an aqueous solution, with a sterile aqueous solution being particularly preferred. However, the components of the kit may be provided as dried powder(s). When reagents and/or components are provided as a dry powder, the powder can be reconstituted by the addition of a suitable solvent. It is envisioned that the solvent may also be provided in another container means.

The container means will generally include at least one vial, test tube, flask, bottle, syringe and/or other container means, into which the nucleic acid formulations are placed, preferably, suitably allocated. The kits may also comprise a second container means for containing a sterile, pharmaceutically acceptable buffer and/or other diluent.

The kits of the present invention will also typically include a means for containing the vials in close confinement for commercial sale, such as, e.g., injection and/or blow-molded plastic containers into which the desired vials are retained.

### V. DEFINITIONS

The term “treatment” or grammatical equivalents encompasses the improvement and/or reversal of the symptoms of disease. “Improvement in the physiologic function” of the eye may be assessed using any of the measurements described herein.

The term “compound” refers to any chemical entity, pharmaceutical, drug, and the like that can be used to treat or prevent a disease, illness, sickness, or disorder of bodily function. Compounds comprise both known and potential therapeutic compounds. A compound can be determined to be therapeutic by screening using the screening methods of the present invention. A “known therapeutic compound” refers to a therapeutic compound that has been shown (e.g., through animal trials or prior experience with administration to humans) to be effective in such treatment. In other words, a known therapeutic compound is not limited to a compound efficacious in the treatment of heart failure.

As used herein, the terms “antagonist” and “inhibitor” refer to molecules, compounds, or agents that inhibit the action of a factor. Antagonists may or may not be homologous to these natural compounds in respect to conformation, charge or other characteristics. Antagonists may have allosteric effects that prevent the action of an agonist. Alternatively, antagonists may prevent the function of the agonist. Antagonists and inhibitors may include proteins, nucleic acids, carbohydrates, small molecule pharmaceuticals or any other molecules that bind or interact with a receptor, molecule, and/or pathway of interest.

As used herein, the term “agonist” refers to molecules or compounds that mimic or promote the action of a “native” or “natural” compound. Agonists may be homologous to these natural compounds in respect to conformation, charge or other characteristics. Agonists may include proteins, nucleic acids, carbohydrates, small molecule pharmaceuticals or any other molecules that interact with a molecule, receptor, and/or pathway of interest.

### VI. EXAMPLES

The following examples are included to further illustrate various aspects of the invention. It should be appreciated by those of skill in the art that the techniques disclosed in the examples which follow represent techniques and/or compositions discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials & Methods

Mice. OX-KO mice (Chemelli et al., 1999), OX1R-KO mice (Mieda et al., 2011), OX2R-KO mice (Willie et al., 2003), OX-Tg mice (Mieda et al., 2004) and littermate WT controls that were backcrossed to the C57BL/6J background for more than ten generations have been previously described. WT and Ob/Ob mice on a C57BL/6J background in the ICV experiments were purchased from Jackson Laboratory. Mice were fed standard chow containing 4% fat ad libitum. All protocols for mouse experiments were approved by the Institutional Animal Care and Use Committee of University of Texas Southwestern Medical Center.

Reagent. OX1R inhibitor (SB-408124) (Langmead et al., 2004) was from Sigma. OX2R inhibitor (compound 1) (Aissaoui et al., 2008) and OX1R2R dual inhibitor (ACT-078573, almorexant) (Brisbare-Roch et al., 2007) were synthesized in the Yanagisawa laboratory. Human orexin-A, orexin-B, and [Ala11, D-Leu15] Orexin-B (OX2R-specific agonist) peptides were from American Peptide Company. Anti-OXR1 (C-19) and anti-leptin (A-20) antibodies were from Santa Cruz Biotechnology. Anti-Ghrelin (clone 1ML-1D7) and anti-orexin-A were from Millipore. Ghrelin siRNA and negative control siRNA were from Sigma.

Bone Analyses. μCT and histomorphometry were performed as described (Wei et al., 2012; Wei et al., 2010). Calcein (20 mg/kg) were injected 2 and 10 days before bone collection. For strength measurement, tibiae were tested by 3-point bending, and a strength parameter (peak load at failure) was assessed with a Test Resources DDL200 axial loading machine outfitted with an Interface SMT1-22 force transducer. Cross-head displacement rate was 0.1 mm/sec. Tests were conducted on the mid-diaphyses with the bones resting on two supports 5 mm apart and the tibial anterior margins facing upward toward the actuator. Serum P1NP and CTX-1 were measured with the Rat/Mouse P1NP EIA kit and the RatLaps™ EIA kit, respectively (Immunodiagnostic Systems) (Wei et al., 2012).

Ex Vivo Bone Marrow Differentiation. Bone marrow cells were cultured for 4 days in MSC media (Mouse MesenCult® Proliferation Kit, StemCell Technologies), then differentiated into osteoblast with a-MEM containing 10% FBS, 5 mM β-glycerophosphate and 100 μg/ml ascorbic acid for 9 days, or differentiated into adipocytes with adipogenesis medium (MesenCult Basal Medium+Mouse MesenCult Adipogenic Stimulatory Supplement, StemCell Technologies) for 7 days (Wei et al., 2012; Wei et al., 2011a; Wei et al., 2011b). For ghrelin knockdown, siRNA was transfected twice with Fugene HD (Roche) the day before and 3 days after differentiation. Osteoclast differentiation was performed as described (Wan et al., 2007; Wei et al., 2010).

Chronic ICV Injection. Mice were single-housed one week before surgery. After anesthetized with 2-3% isoflurane, a cannula (3300PM/SPC; PlasticsOne) was implanted into the right lateral ventricle (0.3 mm posterior from the bregma, 0 9mm lateral from the midline, and 2.4 mm from the surface of skull) using standard sterile stereotactic techniques as described (Funato et al., 2009). An osmotic minipump (model 1004; Alzet) was attached to the cannula and implanted in the subcutaneous space. The OX2R selective agonist ([Ala11, D-Leu15] Orexin-B; American Peptide) (Asahi et al., 2003) or PBS control was continuously injected in the lateral ventricle for 35 days (0.5 nmol/day) before analyses. Ovariectomy or sham operation was performed 3 days before ICV infusion as described (Wei et al., 2011b).

Statistical Analyses. All statistical analyses were performed with Student's t-Test and represented as mean±standard deviation (s.d.) unless stated otherwise. The p values were designated as: *, p<0.05; **, p<0.01; ***, p<0.005; ****, p<0.001; n.s. non-significant (p>0.05).

### Example 2

### Results

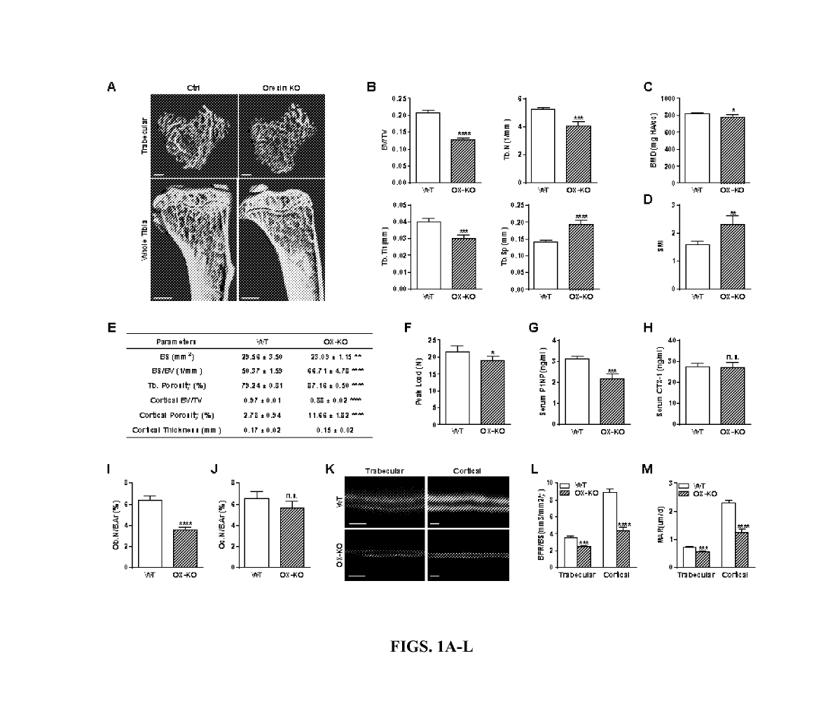

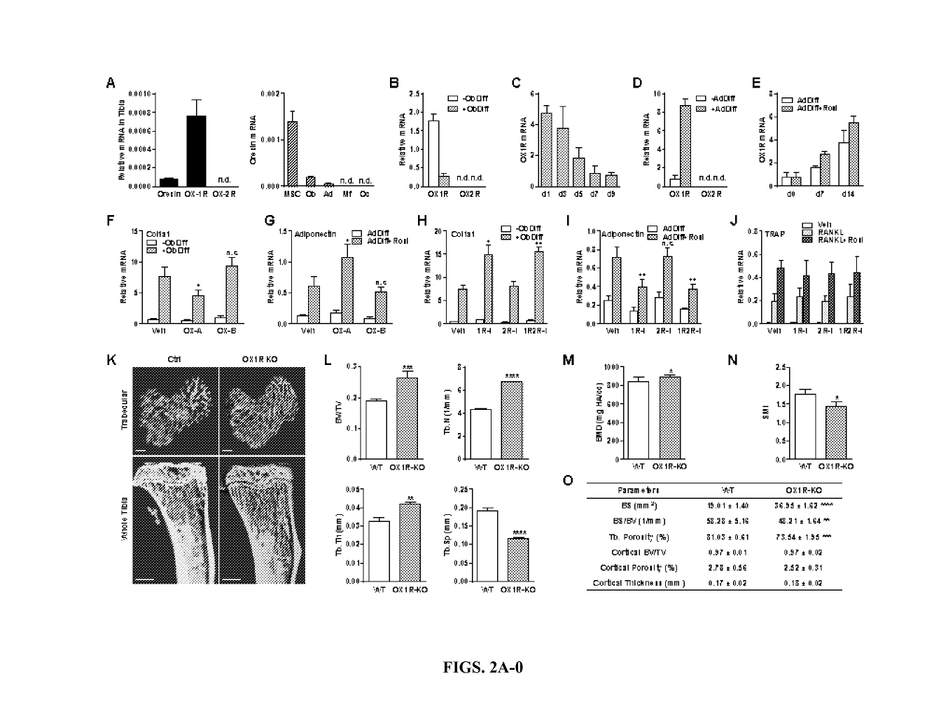

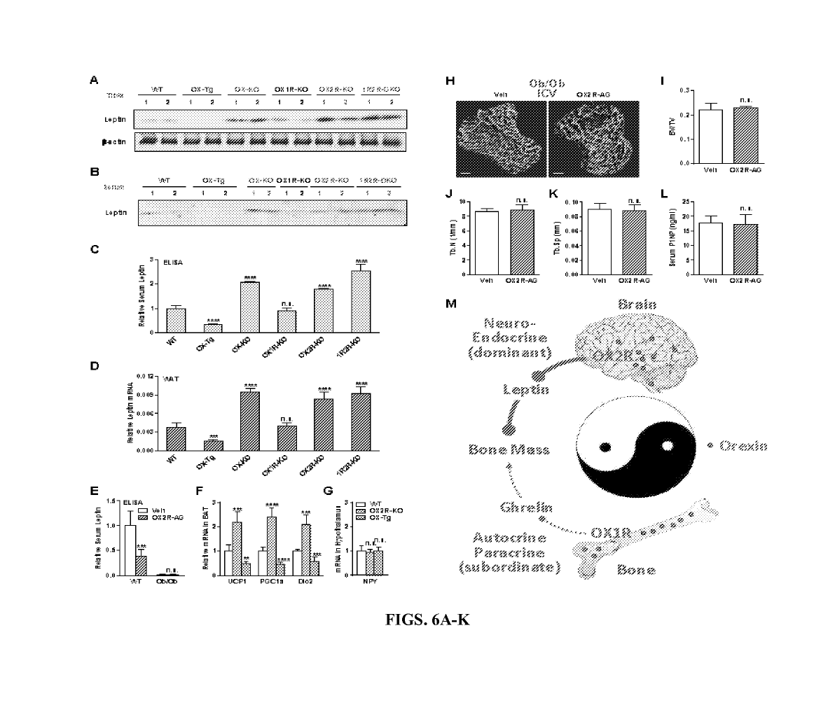

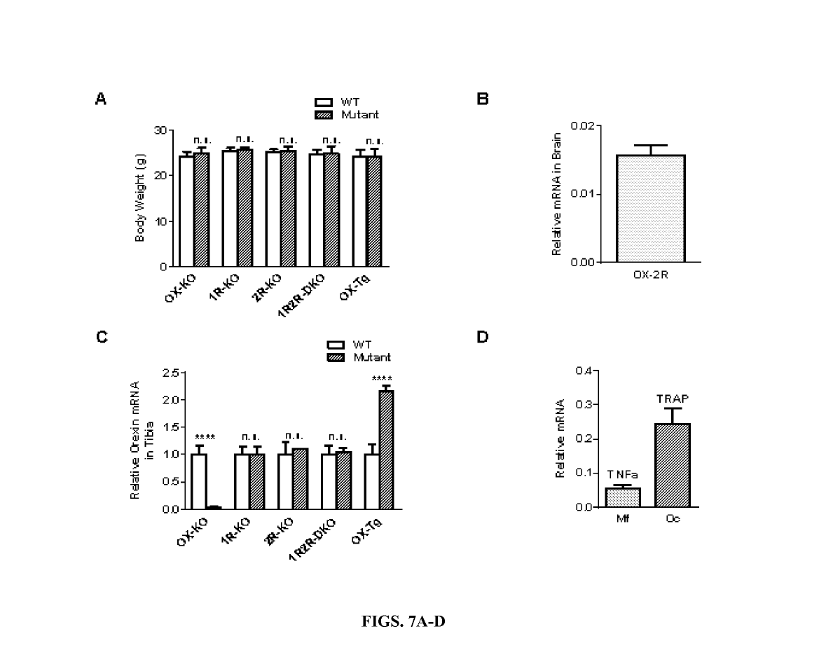

Orexin Deletion Causes Low-Bone-Mass and Decreased Bone Formation. To determine the physiological roles of orexin in skeletal remodeling, the inventorexamined the orexin knockout mice (OX-KO) (Chemelli et al., 1999). MicroCT (μCT) analysis of the trabecular bone in the proximal tibia (FIG. 1A) reveals that the OX-KO mice displayed a low-bone-mass phenotype. The trabecular bone volume/tissue volume ratio (BV/TV) was decreased by 38% in OX-KO compared to WT controls, accompanied by 22% less trabecular number (Tb.N), 24% less trabecular thickness (Tb.Th), 36% greater trabecular separation (Tb.Sp) (FIG. 1B), and 4% lower bone mineral density (BMD) (FIG. 1C). The Structure Model Index (SMI), which quantifies the 3D structure for the relative amount of plates (SMI=0, strong bone) and rods (SMI=3, fragile bone), was 46% higher (FIG. 1D). Consistently, OX-KO mice had lower bone surface (BS), higher BS/BV ratio, and higher trabecular porosity (Tb. Porosity) (FIG. 1E). Moreover, cortical BV/TV was also decreased, leading to higher cortical porosity (FIG. 1E). Consequently, three-point-bending assay shows that the tibiae of OX-KO mice were weaker as the peak load at fracture was 13% lower (FIG. 1F). Consistent with previous reports, body weight of OX-KO mice under chow diet was unaltered (FIG. 7A).

Serum ELISA shows that the bone formation marker N-terminal propeptide of type I procollagen (P1NP) was 30% lower (FIG. 1G), while the bone resorption marker C-terminal telopeptide fragments of the type I collagen (CTX-1) was unaltered (FIG. 1H). Static histomorphometry shows that osteoblast number was decreased (FIG. 1I), whereas osteoclast number was unaltered (FIG. 1J). Dynamic histomorphometry using double calcein labeling shows that OX-KO mice exhibited a lower bone formation rate (BFR/BS) and mineral apposition rate (MAR) in both trabecular bone at metaphysis and cortical bone (FIGS. 1K-M). These results indicate that orexin augments bone mass mainly by promoting bone formation.

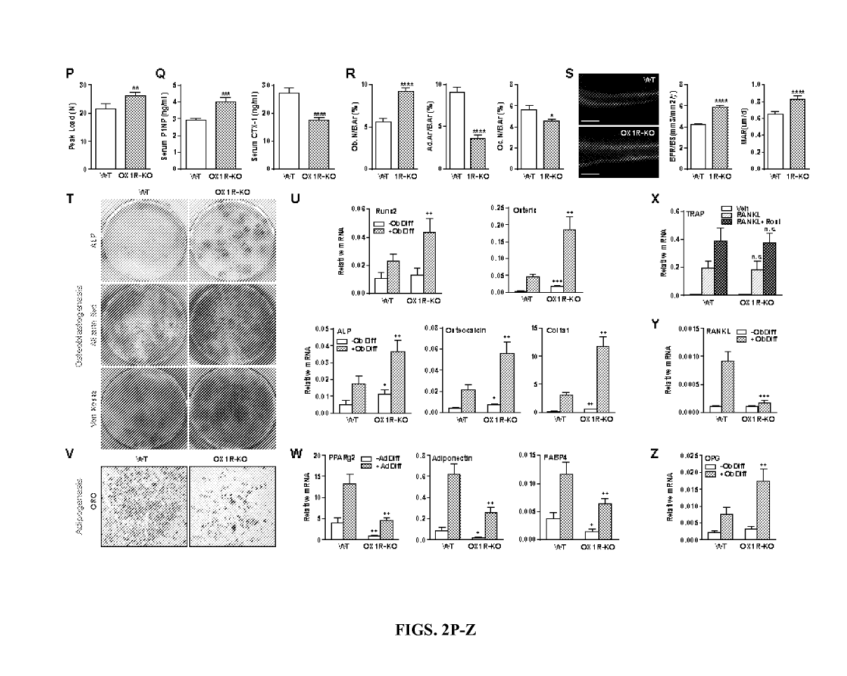

OX1R but not OX2R Regulates Mesenchymal Stem Cell Differentiation. The inventor next investigated whether the orexin regulation of bone mass is mediated by central and/or peripheral actions via OX1R and/or OX2R. Orexin, OX1R and OX2R are all expressed in the brain (Sakurai, 2007), but it was unclear whether they are expressed in bone. She found that orexin and OX1R, but not OX2R, were expressed in mouse tibiae (FIG. 2A, left; FIG. 7B), indicating a specific local orexin regulation via OX1R. Tibial orexin expression, which was absent in OX-KO mice (FIG. 7C), originated from MSCs, osteoblasts and marrow adipocytes but not macrophages or osteoclasts (FIG. 2A, right; FIG. 7D), suggesting an autocrine/paracrine regulation in the mesenchymal lineage.

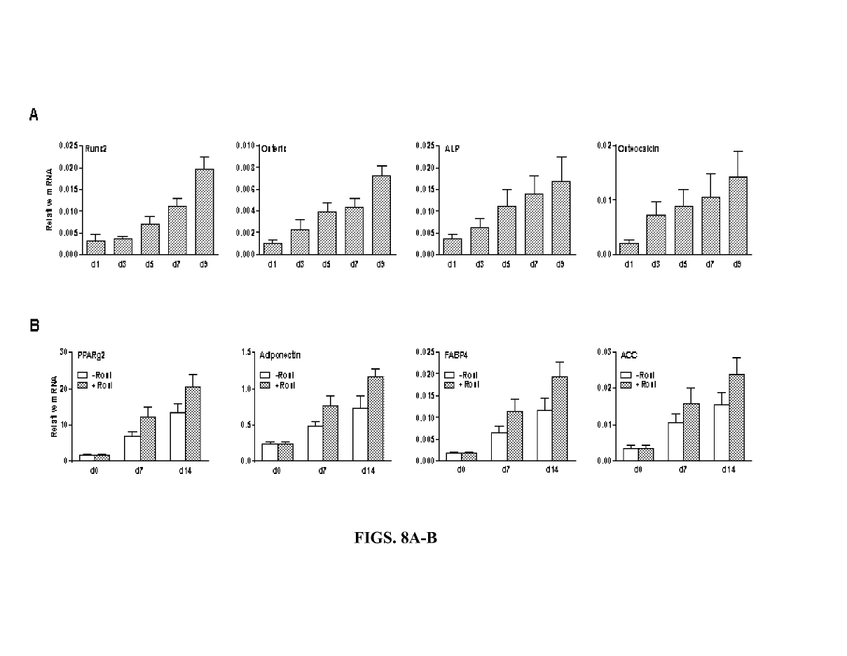

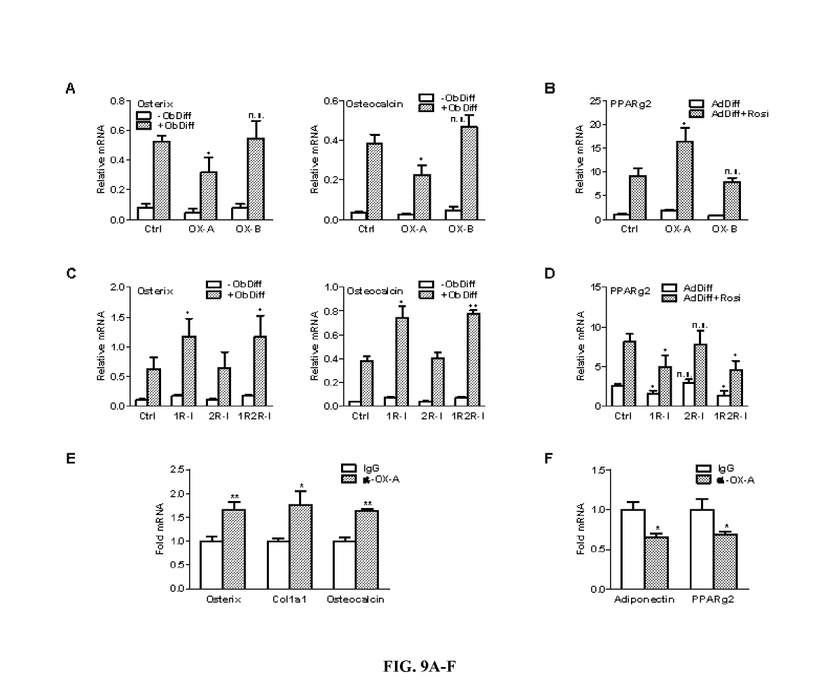

OX1R expression was suppressed during osteoblast differentiation (FIGS. 2B-C) but elevated during adipocyte differentiation (FIGS. 2D-E). Again, OX2R was not expressed in either culture (FIGS. 2B, 2D). Marker gene expression confirmed complete differentiation (FIGS. 8A-B). This indicates that OX1R may be pro-adipogenic and anti-osteoblastogenic. Consistent with this notion, treatment with orexin-A (an agonist for OX1R and OX2R), but not orexin-B (an agonist for mainly OX2R), inhibited osteoblast differentiation (FIG. 2F, FIG. 9A) and enhanced adipocyte differentiation (FIG. 2G, FIG. 9B). Moreover, OX1R inhibitor (SB-408124) or OX1R2R dual inhibitor (ACT-078573) promoted osteoblast differentiation (FIG. 2H, FIG. 9C) and attenuated adipocyte differentiation (FIG. 2I, FIG. 9D), whereas OX2R inhibitor (compound 1) had no effect (FIGS. 2H-I, FIGS. 9C-D). In contrast, these inhibitors did not alter osteoclast differentiation (FIG. 2J), in line with the absence of OX1R and OX2R expression in osteoclasts. An anti-OX-A antibody also increased osteoblast differentiation but decreased adipocyte differentiation (FIG. 9E-F). These results indicate that activation of OX1R inhibits osteoblastogenesis from MSCs by favoring marrow adipogenesis.

OX1R Deletion Causes High-Bone-Mass and Increased Bone Formation. To investigate whether OX1R inhibits bone formation in vivo, the inventor next analyzed OX1R-KO mice (Mieda et al., 2011). μCT shows that OX1R-KO mice exhibited a high-bone-mass phenotype (FIG. 2K). The trabecular bone in OX1R-KO mice had 40% higher BV/TV, 54% higher Tb.N, 29% higher Tb.Th and 39% lower Tb.Sp (FIG. 2L), as well as 5% higher BMD (FIG. 2M) and 19% lower SMI (FIG. 2N). Cortical bone BV/TV, porosity and thickness were not significantly altered (FIG. 20). Moreover, OX1R-KO mice also had stronger bone as the peak load at fracture was 20% higher (FIG. 2P). Serum P1NP was 37% increased (FIG. 2Q, left), whereas serum CTX-1 was 35% decreased (FIG. 2Q, right). Osteoblast number was higher; whereas marrow adipocyte number and osteoclast number were lower (FIG. 2R). BFR/BS and MAR in the trabecular bone at metaphysis was increased (FIG. 2S). Therefore, the high-bone-mass in OX1R-KO mice resulted from a combination of elevated bone formation and reduced bone resorption.

To determine the cellular mechanisms accounting for the higher bone formation in OX1R-KO mice, the inventor next compared bone cell differentiation. Osteoblast differentiation from the marrow MSCs of OX1R-KO mice was enhanced compared to WT control mice, shown by the increased number of alkaline phosphatase⁺ (ALP⁺) cells, alizarin red⁺ cells and von Kossa⁺ cells (FIG. 2T) and the higher expression of osteoblast markers including runx2, osterix, ALP, osteocalcin and collal (FIG. 2U). In contrast, adipocyte differentiation was suppressed, shown by the decreased number of oil-red-o⁺ (ORO⁺) cells (FIG. 2V) and the lower expression of adipocyte markers including PPARγ2, adiponectin and FABP4 (FIG. 2W). These results are consistent with the findings in FIGS. 2B-I, demonstrating that OX1R is anti-osteoblastogenic but pro-adipogenic.

Since bone resorption was also decreased in OX1R-KO mice, the inventor next compared osteoclastogenesis. When stimulated with the same concentration of RANKL, the bone marrow of OX1R-KO mice differentiated into osteoclasts to a similar extent as WT mice (FIG. 2X), consistent with the results in FIG. 2J. This indicates that the reduced bone resorption was caused by a non-cell-autonomous effect. The inventor found that RANKL expression was lower (FIG. 2Y) whereas OPG expression was higher (FIG. 2Z) in OX1R-KO osteoblast differentiation cultures compared to WT cultures, indicating that the reduced bone resorption in OX1R-KO mice was mediated by a decreased RANKL/OPG ratio.

Together, these findings unexpectedly present a dichotomy where OX1R-KO mice exhibit a high-bone-mass that is the opposite of the low-bone-mass in the OX-KO mice, indicating that additional mechanisms may dominate over OX1R in orexin regulation of bone. Although OX2R is not expressed in bone, a possibility exists that OX2R may mediate a central regulation by orexin. Thus, the inventor next investigated the consequences of OX2R deletion.

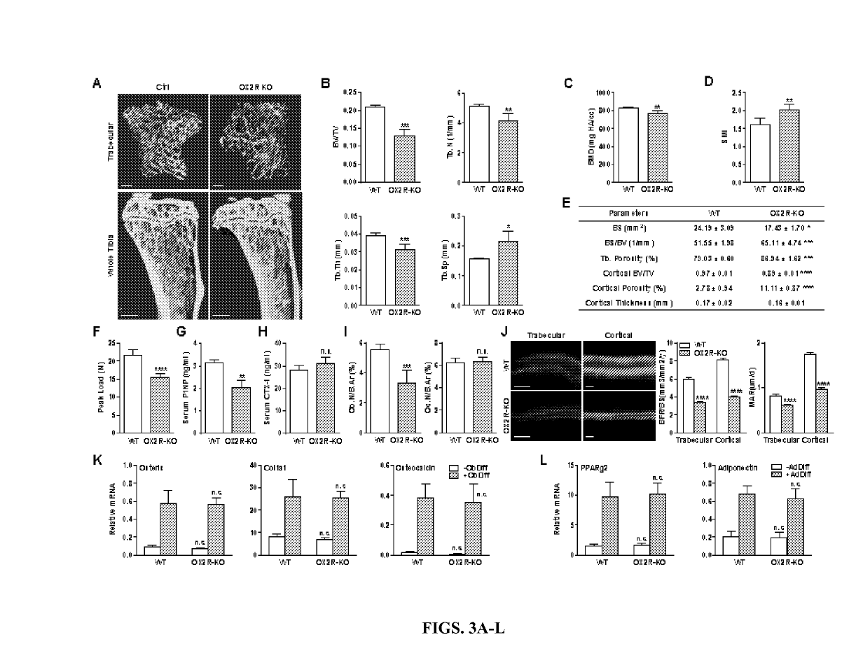

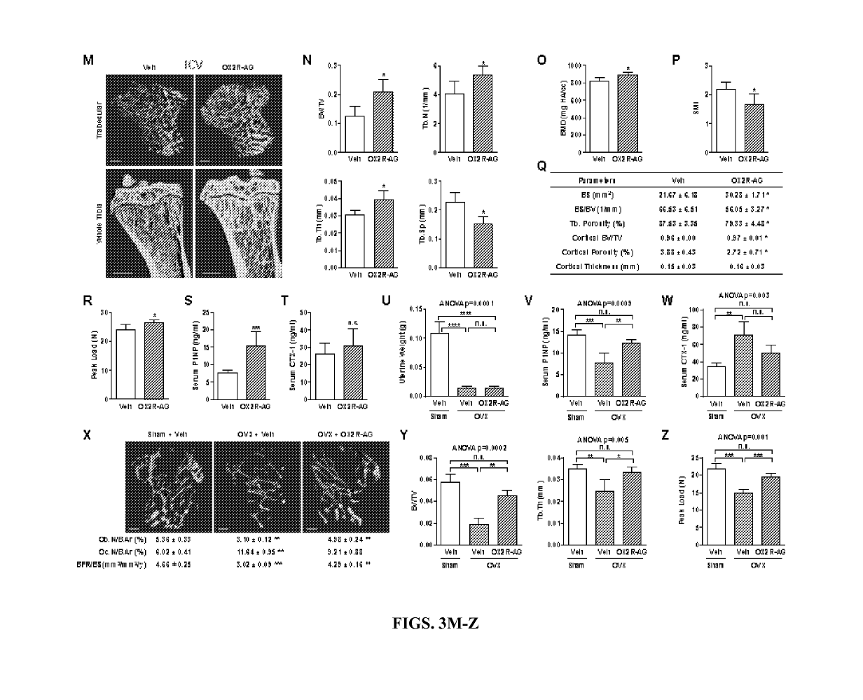

OX2R Deletion Causes Low-Bone-Mass and Decreased Bone Formation. μCT shows that OX2R-KO mice (Willie et al., 2003) exhibited a low-bone-mass (FIGS. 3A-E) as in the OX-KO mice, leading to a reduced peak load at fracture (FIG. 3F). P1NP and osteoblast number were decreased, whereas CTX-1 and osteoclast number were unaltered (FIGS. 3G-I). Consistently, BFR/BS and MAR were reduced in both males (FIG. 3J) and females (FIGS. 11A-D). In agreement with the lack of OX2R expression in bone, osteoblast and adipocyte (FIGS. 2A, 2B, 2D), marrow MSCs from OX2R-KO mice displayed normal capacity to differentiate into osteoblasts (FIG. 3K) and adipocytes (FIG. 3L). These results show that OX2R deletion causes low-bone-mass that recapitulates the bone defects in OX-KO mice, due to a reduction in bone formation via potentially a central mechanism.

Central administration of an OX2R-selective agonist augments bone mass. To further test the hypothesis that OX2R acts centrally to regulate skeletal homeostasis, the inventor performed intracerebroventricular (ICV) injection. An OX2R selective agonist (OX2R-AG) [Ala11, D-Leu15] orexin-B (Asahi et al., 2003) was continuously infused in the lateral ventricles of WT mice at 0.5 nmol/day for 35 days as described (Funato et al., 2009). μCT shows that OX2R-AG remarkably enhanced bone mass (FIG. 3M), leading to 66% higher BV/TV, 32% higher Tb.N, 30% higher Tb.Th and 33% lower Tb.Sp (FIG. 3N); as well as 8% higher BMD (FIGS. 3O) and 25% lower SMI (FIG. 3P). The cortical bone had 1% higher BV/TV and 30% less porosity (FIG. 3Q). Moreover, OX2R-AG also increased bone strength as the peak load at fracture was 11% higher (FIG. 3R). P1NP was 100% increased (FIG. 3S), whereas CTX-1 was unaltered (FIG. 3T). These results suggest that central activation of OX2R enhances bone formation and augments bone mass.

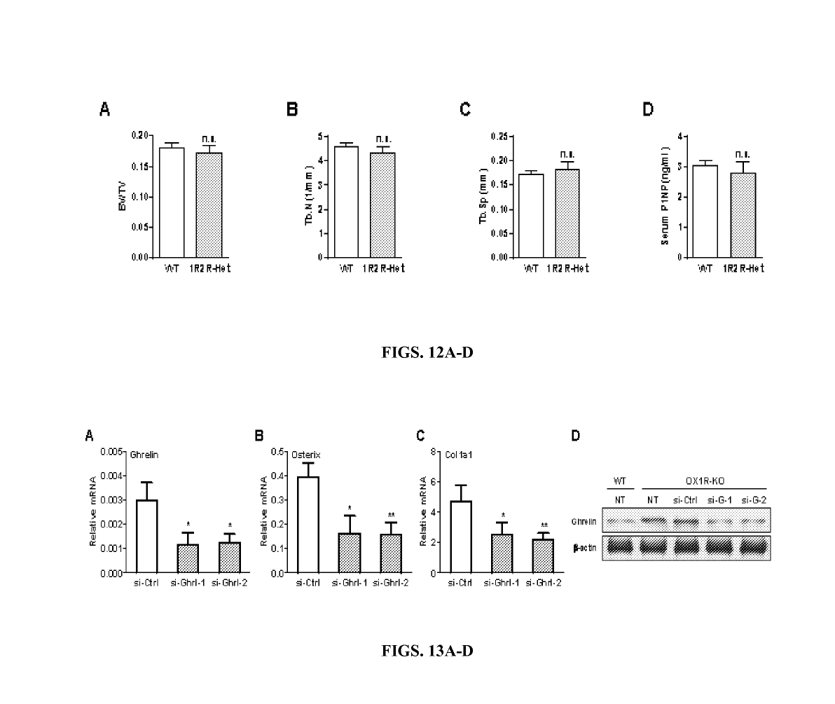

The inventor next investigated whether OX2R-AG ICV injection can serve as a therapeutic strategy to treat postmenopausal osteoporosis in an ovariectomy (OVX) mouse model. Three days after surgery, the inventor began the ICV infusion at 0.5 nmol/day for 35 days. Uterine weight was reduced by ˜87% in all ovariectomized mice compared to sham controls, indicating effective estrogen depletion (FIG. 3U). OVX-mediated reduction in P1NP was significantly abolished by OX2R-AG (FIG. 3V); whereas OVX-mediated increase in CTX-1 was not significantly altered (FIG. 3W). Moreover, OVX-mediated reduction in osteoblast number and bone formation rate was also rescued by OX2R-AG (FIG. 3X). Consequently, OVX-induced bone loss was attenuated in OX2R-AG treated mice (FIGS. 3X-Z). This bone anabolic effect of OX2R-AG was abolished in OX2R-KO mice (FIGS. 12A-D), confirming that it is OX2R-dependent. These results suggest that central activation of OX2R may be a strategy to ameliorate bone degenerative diseases such as osteoporosis.

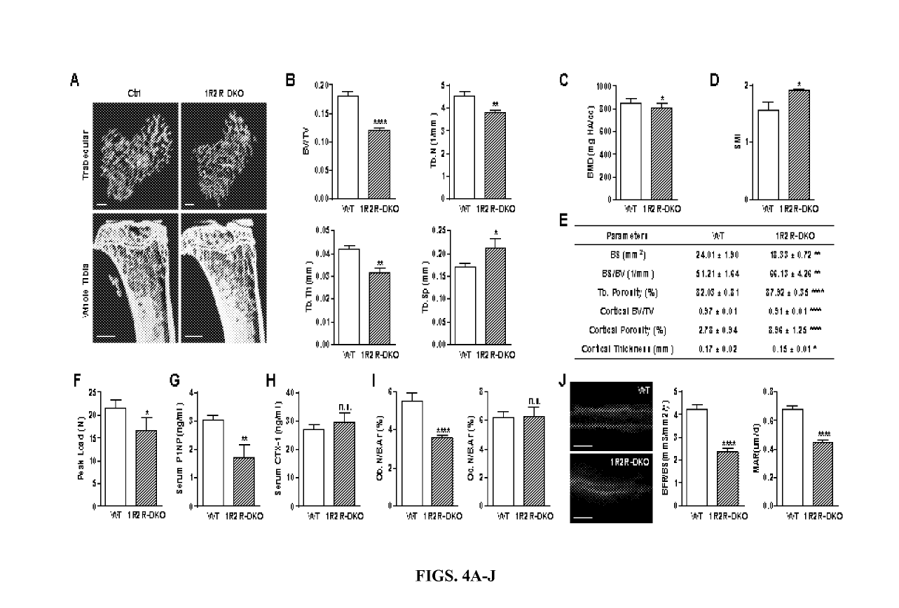

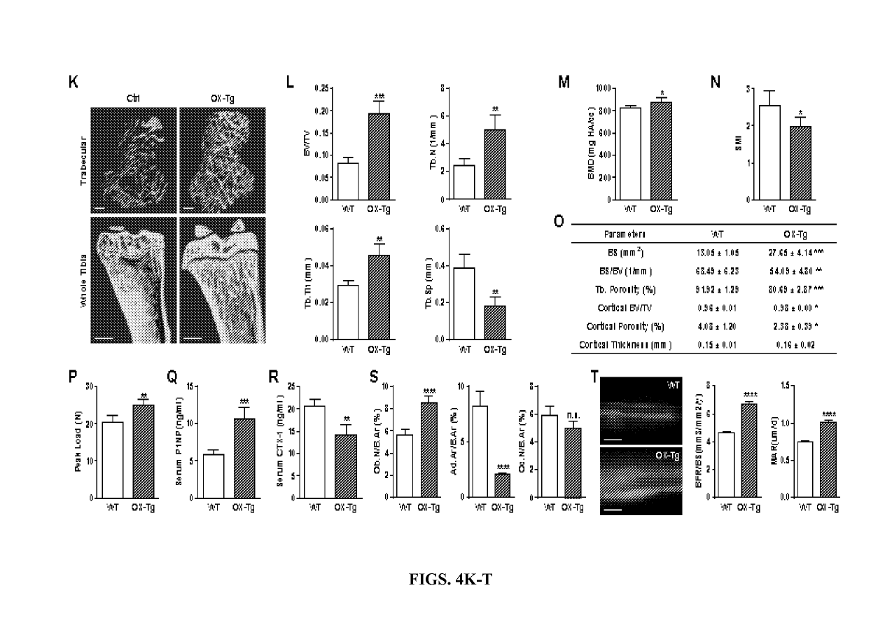

Central Action is Dominant over Peripheral Action in Orexin Regulation of Bone. The observation that OX-KO mice exhibited a similar bone phenotype as OX2R-KO mice rather than OX1R-KO mice suggests that the OX2R-mediated central control is dominant over the OX1R-mediated peripheral regulation. To further test this hypothesis, the inventor analyzed the OX1R2R double knockout mice (1R2R-DKO). μCT shows that 1R2R-DKO mice also exhibited a low-bone-mass (FIGS. 4A-F). P1NP was decreased whereas CTX-1 was unaltered (FIGS. 4G-H). Osteoblast number, BFR/BS and MAR were reduced whereas osteoclast number was unaltered (FIGS. 4I-J). OX1R2R double heterozygous mice did not exhibit any significant bone phenotype (FIGS. 13A-D). These results indicate that simultaneous deletion of both OX1R and OX2R presents an OX2R-null phenotype.

As a complimentary gain-of-function approach, the inventor examined the effects of global orexin over-expression by analyzing the CAG/orexin-transgenic mice (OX-Tg) (Mieda et al., 2004). The pattern of orexin over-expression in the OX-Tg mice has been described (Funato et al., 2009; Mieda et al., 2004): ectopic orexin immunoreactivity was detected in several CNS regions as well as in a limited set of peripheral tissues, including thyroid gland, adrenal cortex and some pancreatic islets, but not in other metabolic tissues such as brown and white adipose, liver, or skeletal muscle. In agreement, the percentage of brown adipose tissue weight in body weight was unaltered in OX-Tg mice (0.306±0.017%) compared with WT littermate controls (0.302±0.019%). Tibial orexin expression was elevated by 115% in the OX-Tg mice compared with controls (FIG. 7B). μCT shows that OX-Tg mice exhibited high-bone-mass (FIGS. 4K-P). Bone formation was elevated as P1NP was 83% higher (FIG. 4Q). Interestingly, chronic global orexin over-expression also suppressed bone resorption as CTX-1 was 31% lower (FIG. 4R). Osteoblast number, BFR/BS and MAR were higher, adipocyte number was lower, and osteoclast number was not significantly altered (FIGS. 4S-T). Together, the results that bone mass is reduced in OX-KO and 1R2R-DKO mice but enhanced in OX-Tg mice strongly supports the notion that the OX2R-mediated central regulation is dominant over the OX1R-mediated peripheral modulation of bone cell differentiation.

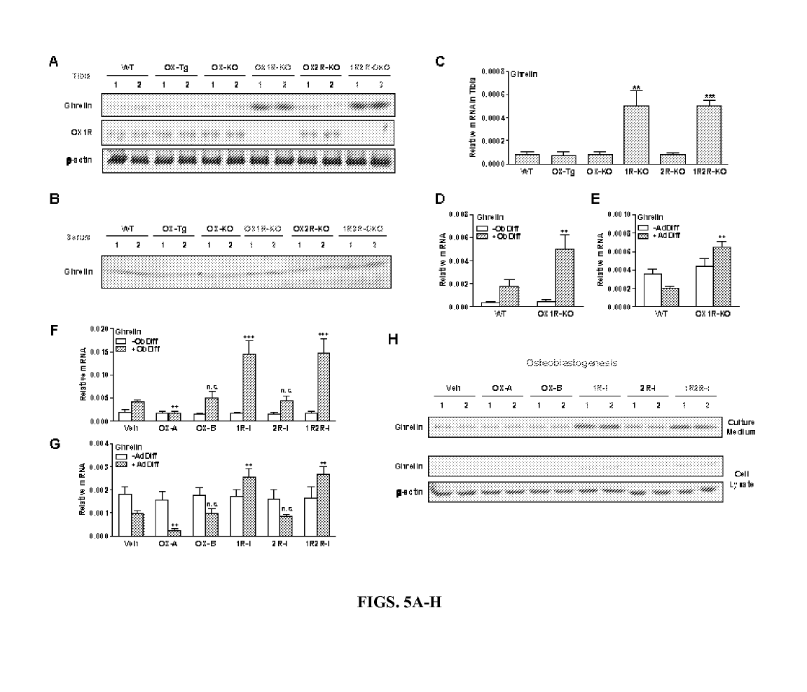

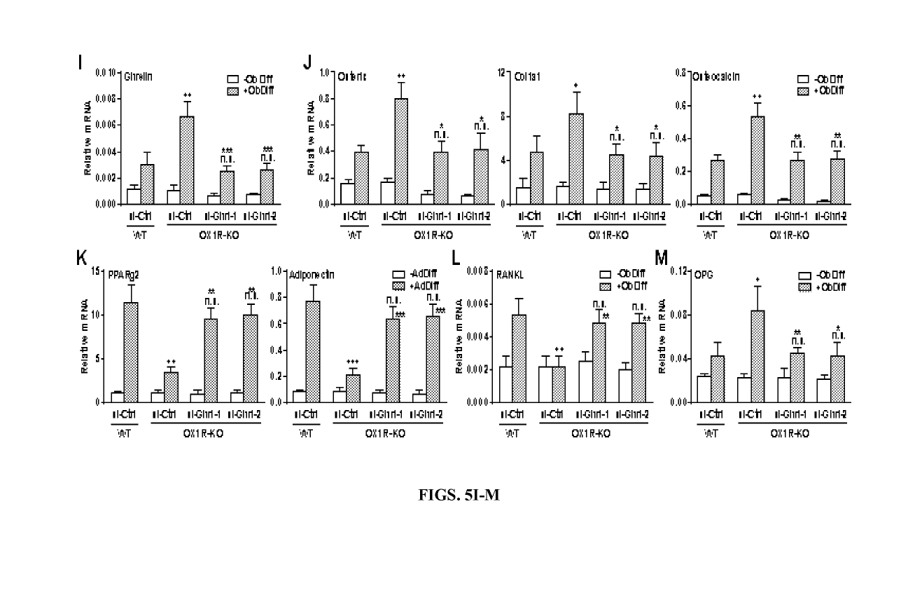

OX1R Inhibits Osteoblastogenesis by Suppressing Local Ghrelin Expression. The inventor next set out to elucidate the molecular mechanisms underlying the local and central bone regulation by orexin. She found that the expression of ghrelin protein was markedly up-regulated in the tibiae of OX1R-KO and 1R2R-DKO mice (FIG. 5A). In contrast, the level of serum ghrelin protein, which largely derives from the stomach, was unaltered (FIG. 5B). This suggests that OX1R regulation of ghrelin expression occurs locally in the bone. Indeed, ghrelin mRNA was also higher in the tibiae of OX1R-KO and 1R2R-DKO mice (FIG. 5C).

Ghrelin expression was induced during osteoblast differentiation, which was enhanced in the culture from OX1R-KO mice compared to WT controls (FIG. 5D). In contrast, ghrelin expression was reduced during adipocyte differentiation, which was also elevated in the culture from OX1R-KO mice compared to WT controls (FIG. 5E). In line with these findings, the ghrelin induction during osteoblast differentiation was abolished by OX-A treatment but potentiated by OX1R inhibitor or OX1R2R dual inhibitor (FIG. 5F). Ghrelin expression in adipocyte differentiation cultures was also inhibited by OX-A, but enhanced by OX1R inhibitor or OX1R2R dual inhibitor (FIG. 5G). Furthermore, ghrelin secretion was elevated by OX1R inhibitor or OX1R2R dual inhibitor in both osteoblast (FIG. 5H) and adipocyte (not shown) cultures. Previous studies have shown that ghrelin promotes osteoblastogenesis (Delhanty et al., 2006; Fukushima et al., 2005; Kim et al., 2005; Maccarinelli et al., 2005). Thus, these results indicate that OX1R inhibition of osteoblast differentiation may be mediated by the suppression of local ghrelin expression in bone.

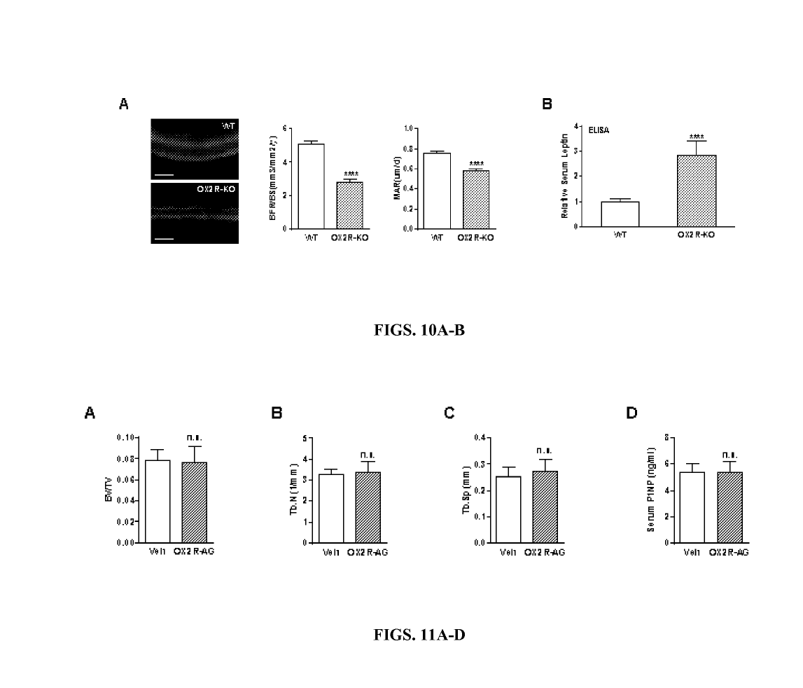

To further investigate whether ghrelin up-regulation was required for the pro-osteoblastogenic and anti-adipogenic effects of OX1R deletion, the inventor performed ghrelin siRNA knockdown experiments. Marrow MSCs from WT or OX1R-KO mice were transfected with ghrelin siRNA (si-Ghrl) or control siRNA (si-Ctrl) before differentiation. Ghrelin knockdown in WT cells decreased osteoblast differentiation (FIGS. 10A-C), supporting the pro-osteoblastogenic role of ghrelin. Importantly, ghrelin knockdown in OX1R-KO cells to a level similar to WT cells (FIG. 5I, FIG. 10D) abolished their ability to increase osteoblastogenesis, decrease adipogenesis, or alter RANKL and OPG expression (FIGS. 5J-M). These findings indicate that osseous ghrelin is an essential mediator of the local bone regulation by OX1R.

OX2R Augments Bone Formation by Suppressing Serum Leptin Level. Leptin suppresses bone formation, and serum leptin level is a critical determinant of bone mass. The inventor found that leptin protein levels in both bone and serum were elevated in OX-KO, OX2R-KO and OX1R2R-DKO mice but reduced in OX-Tg mice (FIGS. 6A-C). Leptin mRNA showed a similar pattern in white adipose tissue (WAT) (FIG. 6D) but was undetectable in bone (not shown), indicating that the changes in leptin protein in bone mainly originated from peripheral fat via circulation. Consistent with the genetic evidence, ICV injection of an OX2R agonist also decreased serum leptin in WT mice (FIG. 6E). These results indicate that orexin may enhance bone formation by suppressing leptin levels.

Leptin has been shown to decrease trabecular bone mass at least in part by activating the sympathetic nerves (Ducy et al., 2000; Elefteriou et al., 2004). Interestingly, recent studies suggest that leptin paradoxically increases cortical bone mass at least in part by down-regulating hypothalamic expression of NPY, a neuropeptide that causes bone loss (Baldock et al., 2002; Lee and Herzog, 2009; Wong et al., 2013). Since both trabecular and cortical bone mass was decreased in OX-KO and OX2R-KO mice but increased in OX-Tg mice, the inventor next examined the sympathetic outflow and hypothalamic NPY expression in these mice. Expression analyses of UCP1, PGC1α and Dio2 in brown adipose tissue (BAT) show that the sympathetic tone was increased in OX2R-KO mice but decreased in OX-Tg mice (FIG. 6F). In contrast, expression of NPY in hypothalamus was unaltered (FIG. 6G). These results suggest that orexin augments bone mass mainly by suppressing leptin activation of the sympathetic nerves.

To further elucidate whether leptin is required for OX2R regulation of bone formation, the inventor performed ICV injection of OX2R-AG in Ob/Ob mice that lack functional leptin protein (Zhang et al., 1994) (FIG. 6E). In contrast to WT mice (FIGS. 3M-T), the bone enhancing effects of OX2R-AG was completely abolished in Ob/Ob mice, as OX2R-AG was no longer able to increase BV/TV, Tb.N, Tb.Sp or P1NP (FIGS. 6H-L). These results indicate that leptin is a critical mediator of the central bone regulation by OX2R.

### Example 3

### Discussion

This study has identified orexin as a critical yet previously unrecognized regulator of bone mass accrual that functions via a yin-yang dual mechanism (FIG. 6M). On one hand, orexin activation of OX2R in the brain centrally enhances bone formation by lowing circulating leptin level. On the other hand, orexin activation of OX1R in the bone locally suppresses bone formation and enhances bone resorption by lowering osseous ghrelin expression. Importantly, the central action is dominant over local action so that systemic orexin over-expression increases bone mass whereas complete deletion of orexin or orexin receptors decreases bone mass. It is remarkable how orexin achieves a physiological balance in the regulation of skeletal homeostasis by differentially utilizing two different receptors at distinct anatomic sites.

Orexin deficiency in human causes behavior abnormalities including sleep and mood disorders. Both OX-KO and OX2R-KO mice exhibit a narcolepsy phenotype, which is characterized by daytime sleepiness that is accompanied by a sudden loss of muscle tone known as cataplexy, often after laughter or excitement (Chemelli et al., 1999; Lin et al., 1999). Orexin is undetectable in most human narcolepsy patients (Nishino et al., 2000). Interestingly, older women with sleep disorders are reported to suffer from greater risk of osteoporotic fractures (Stone et al., 2006). Moreover, orexin deficiency and sleep disorders are also frequently associated with major mood disorders (MMD), especially depression (Allard et al., 2004; Brundin et al., 2007). In humans, orexin A levels in amygdala are maximal during positive emotion but minimal during depression, suggesting that boosting orexin function could elevate mood (Blouin et al., 2013). In mice, orexin neurons are also maximally active during performance of rewarded behaviors; OX-KO mice are deficient in conducting rewarded behaviors; and OX2R-KO mice display increased behavioral despair, indicating a similar involvement of orexin in positive reinforcement (Borgland et al., 2009; McGregor et al., 2011; Scott et al., 2011). In humans, depression is associated with low bone mass and increased incidence of osteoporotic fractures (Bab and Yirmiya, 2010). A study using a mouse stress model shows that depression induces bone loss by inhibiting bone formation via the stimulation of the sympathetic nervous system (Yirmiya et al., 2006). However, the neural circuitry underlying the connection of narcolepsy, depression and bone loss is not well understood. These findings that OX-KO and OX2R-KO mice exhibit lower bone mass and higher leptin levels provide a potential mechanism for the increased fracture risk in narcolepsy and depression.

Orexin deficiency is also associated with metabolic abnormalities including obesity and hypophagia (Sakurai, 2007; Sakurai and Mieda, 2011). The obese phenotype in young mice occurs only under high-fat-diet feeding, but not under chow-diet-feeding (Funato et al., 2009; Sellayah et al., 2011), and at least in part due to decreased energy expenditure and impaired development of BAT (Sellayah et al., 2011), which have recently been reported to promote bone formation (Rahman et al., 2013). Interestingly, the decreased energy expenditure in OX-KO mice was caused by an OX1R-dependent direct BAT differentiation defect rather than defects in sympathetic nervous system (Sellayah et al., 2011). Thus, even if leptin and sympathetic tone are increased in OX-KO mice due to a central effect, leading to decreased bone mass, energy expenditure is still lower due to the lack of BAT. Consistent with this notion, these results show that leptin levels are only elevated in OX2R-KO but not OX1R-KO mice, and reduced by ICV delivery of OX2R-agonist (FIGS. 6A-E), indicating that orexin suppresses leptin via a central regulation. Of note, the inventor found that orexin and orexin receptors can regulate bone remodeling in the absence of body weight change under chow-diet (FIG. 7A). In addition, orexin expression in the brain (Mieda et al., 2011; Willie et al., 2003) and tibiae (FIG. 7B) are unaltered in the receptor knockout mice. Furthermore, the inventor's previous study show that OX-Tg mice have no ectopic orexin protein expression in BAT and WAT (Funato et al., 2009), suggesting that orexin regulation of bone can be independent from its regulation of BAT. Nonetheless, it is possible that these other metabolic and behavior changes may indirectly contribute to the skeletal phenotype observed in orexin and orexin receptor knockout mice.

Global knockout mice have the advantage of revealing the net effects of loss-of-function, including both cell-autonomous and systemic/non-cell-autonomous effects. Indeed, the inventor's findings uncover OX1R in the osteoblasts and OX2R in the brain as two potential mechanisms contributing to the peripheral and central bone regulation by orexin, respectively. The inventor have identified a cell-autonomous role of orexin and OX1R in osteoblast differentiation, and thus provided a key mechanism for how OX1R regulates bone. It is possible that other non-cell autonomous effects may also contribute to the bone phenotype of OX1R-KO mice. Although ICV injection delivers OX2R agonist to several CNS regions, it is known that endogenous orexin peptides are also widely projected in the brain (Sakurai, 2007; Sakurai and Mieda, 2011). The finding that the bone anabolic effects of OX2R agonist is abolished in the Ob/Ob mice indicates that leptin may be an essential mediator of the central bone regulation by orexin and OX2R; although other indirect mechanisms in Ob/Ob mice, such as the altered metabolism, may also contribute to their resistance to OX2R agonist. Future studies to generate and characterize conditional knockout mice for orexin and orexin receptors using flox mice and cre drivers specific for MSCs, pre-osteoblasts, mature osteoblasts, and specific CNS regions will further delineate the functional requirement of each tissue and cell type.

The inventor's in vivo genetic studies using OX1R knockout mice, in combination with the inventor's in vitro bone marrow osteoblast differentiation assays using orexin peptides and orexin receptor inhibitors, demonstrate that OX1R suppresses osteoblast differentiation and bone formation. Orexin regulation of osteoblastogenesis has also been implicated in a previous study using rat calvarial osteoblast-like cells (Ziolkowska et al., 2008). The inventor found that OX1R inhibits osteoblast differentiation by specifically reducing osseous ghrelin expression without altering circulating ghrelin levels (FIGS. 5A-M), suggesting that distinct mechanisms may account for the regulation of ghrelin expression in bone and stomach. The ghrelin siRNA knockdown experiments (FIGS. 5A-M), and previous pharmacological experiments (Delhanty et al., 2006; Fukushima et al., 2005; Kim et al., 2005; Maccarinelli et al., 2005), show that ghrelin promotes osteoblastogenesis. Moreover, pharmacological studies also show that ghrelin stimulates growth and appetite. Interestingly however, it is reported that ghrelin knockout mice are normal with unaltered body weight, food intake and bone density (Sun et al., 2003). A possible explanation is that developmental compensation in the knockout mice may mask the physiological role of ghrelin.

The elucidation of the intricate mechanisms underlying orexin regulation of bone also presents exciting opportunities for the treatment of bone degenerative diseases such as osteoporosis. First, OX2R-specific agonists hold tremendous potential as bone anabolic therapeutics. Second, OX1R-specific antagonists may present anabolic and anti-catabolic dual benefits to enhance bone formation and suppress bone resorption. Interestingly, OX2R activation also confers resistance to obesity and diabetes (Funato et al., 2009; Kotz et al., 2012), hence OX2R agonists may promote metabolic and skeletal fitness simultaneously. This prospect is even more exciting in light of the bone loss side effects observed in the current drugs or drug candidates for obesity/diabetes such as rosiglitazone (Bilezikian et al., 2013; Grey, 2009; Home et al., 2009; Kahn et al., 2006; Kahn et al., 2008; Zinman et al., 2010) and fibroblast growth factor 21 (FGF21) (Wan, 2013; Wei et al., 2012).

Several pharmaceutical companies including Merck and GlaxoSmithKline have developed small molecule orexin antagonists for the treatment of insomnia These include OX1R antagonists, OX2R antagonists and OX1R2R dual antagonists such as almorexant (ACT-078573) (Brisbare-Roch et al., 2007) and suvorexant (MK-4305) (Cox et al., 2010; Willyard, 2012). Suvorexant is under FDA review after completion of Phase III clinical trials (Mieda and Sakurai, 2013; Willyard, 2012). These findings suggest that OX1R-specific antagonist may be bone protective whereas OX2R antagonists and OX1R2R dual antagonists may risk bone loss.

More fundamentally, the inventor's discovery of orexin as a key regulator of skeletal homeostasis provides crucial insight to the understanding of how bone remodeling is controlled by neuronal and endocrine mechanisms. It also raises a provocative question of how skeletal physiology may crosstalk with sleep/wake, fast/feeding, energy store/expenditure cycles, as well as reward, addiction, anxiety and motivation behaviors, via the common orexin pathway in vertebrates.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods, and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### VII. REFERENCES

The following references, to the extent that they provide exemplary procedural or other details supplementary to those set forth herein, are specifically incorporated herein by reference.
Aissaoui et al., Bioorganic & medicinal chemistry letters 18:5729-5733, 2008.
Allard et al., Neuropeptides 38:311-315, 2004.
Asahi et al., Bioorganic & medicinal chemistry letters 13:111-113, 2003.
Ashton et al., Bone, 6:313-319,1985.
Aubin, Biochem. Cell Biology, 76:899-910,1998.
Bab et al., Annals of the New York Academy of Sciences 1192:170-175, 2010.
Baldock et al., The Journal of clinical investigation 109:915-921, 2002.
Bianco et al., Nature medicine 19:35-42, 2013.
Bilezikian et al., The Journal of clinical endocrinology and metabolism 98:1519-1528, 2013.
Bleiberg, Connect Tissue Res., 14:121-127,1985.
Blouin et al., Nature communications 4:1547, 2013.
Borgland et al., The Journal of neuroscience: the official journal of the Society for Neuroscience 29:11215-11225, 2009.
Brisbare-Roch et al., Nature medicine 13:150-155, 2007.
Brundin et al., Eur Neuropsychopharmacol 17:573-579, 2007.
Chemelli et al., Cell 98:437-451, 1999.
Cox et al., Journal of medicinal chemistry 53:5320-5332, 2010.
Delhanty et al., The Journal of endocrinology 188:37-47, 2006.
Ducy et al., Cell 100:197-207, 2000.
Elefteriou et al., Proceedings of the National Academy of Sciences of the United States of America 101:3258-3263, 2004.
Friedenstein et al., Exp. Hematol., 10:217-227,1982.
Friedenstein et al., Transplantation, 6:230-247,1968.
Fukushima et al., Ghrelin directly regulates bone formation. Journal of bone and mineral research: the official journal of the American Society for Bone and Mineral Research 20:790-798, 2005.
Funato et al., Cell metabolism 9:64-76, 2009.
Grey, Diabetes Obes Metab 11:275-284, 2009.
Gronthos et al., Blood, 84:4164-4173,1994.
Gronthos et al., J. Bone Min. Res., 14:47-56,1999.
Hara et al., Neuron 30:345-354, 2001.
Home et al., Lancet 373:2125-2135, 2009.
Jaiswal et al., J. Biol. Chem., 275:9645-9652, 2000.
Kadiyala et al., Cell Transplantation, 6:125-134,1997.
Kahn et al., Diabetes Care 31:845-851, 2008.
Kahn et al., N Engl J Med 355:2427-2443, 2006.
Kale et al., Nat. Biotech., 18:954-958, 2000.
Kim et al., Bone 37:359-369, 2005.
Kotz et al., Annals of the New York Academy of Sciences 1264:72-86, 2012.
Langmead et al., British journal of pharmacology 141:340-346, 2004.
Lin et al., Cell 98:365-376, 1999.
Maccarinelli et al., The Journal of endocrinology 184:249-256, 2005.
McGregor et al., The Journal of neuroscience: the official journal of the Society for Neuroscience 31:15455-15467, 2011.
Mieda et al., Proceedings of the National Academy of Sciences of the United States of America 101:4649-4654, 2004.
Mieda et al., The Journal of neuroscience: the official journal of the Society for Neuroscience 31:6518-6526, 2011.
Nishino et al., Lancet 355:39-40, 2000.
Novack and Teitelbaum, Annu Rev Pathol 3:457-484, 2008.
Petite et al., Nat. Biotech., 18:959-963, 2000.
Peyron et al., Nature medicine 6:991-997, 2000.
Phinney et al., J. Cellular Biochem., 75:424-436,1999.
Pittenger et al., Science, 284:143-147,1999.
Rahman et al., Inducible Brown Adipose Tissue, or Beige Fat, Is Anabolic for the Skeleton. Endocrinology, 2013.
Reddi and Huggins, Proc. Natl. Acad. Sci. USA, 69:1601-1605,1972.
Remington's Pharmaceutical Sciences, 15^{th} ed., pages 1035-1038 and 1570-1580, Mack
Publishing Company, Easton, Pa., 1980.
Rosen, Cell metabolism 7:7-10, 2008.
Sakurai and Mieda, Trends Pharmacol Sci 32:451-462, 2011.
Sakurai, Nat Rev Neurosci 8:171-181, 2007.
Scott et al., Behav Brain Res 222:289-294, 2011.
Sellayah et al., Cell metabolism 14:478-490, 2011.
Stone et al., J Am Geriatr Soc 54:1177-1183, 2006.
Sun et al., Molecular and cellular biology 23:7973-7981, 2003.
U.S. Pat. No. 5,972,703
U.S. Pat. No. 5,972,900
Wan et al., Nat Med 13:1496-1503, 2007.
Wan, The International Journal of Biochemistry & Cell Biology 45:546-549, 2013.
Wei et al., Cell Metab 11:503-516, 2010.
Wei et al., Mol Cell Biol 31:4692-4705, 2011b.
Wei et al., Mol Cell Biol 31:4706-4719, 2011a.
Wei et al., Proc Natl Acad Sci USA 109:3143-3148, 2012b.
Wei et al., The Journal of cell biology 197, 509-521, 2012a.
Willie et al., Neuron 38:715-730, 2003.
Willyard, Nature medicine 18:996, 2012.
Wong et al., Journal of bone and mineral research: the official journal of the American Society for Bone and Mineral Research 28:886-898, 2013.
Zhang et al., Nature 372:425-432, 1994.
Zinman et al., J Clin Endocrinol Metab 95:134-142, 2010.
Ziolkowska et al., Int J Mol Med 22:749-755, 2008.

What is claimed:

1. A method of increasing bone mass and/or volume in a subject comprising:
(a) identifying a patient in need of increased bone mass and/or volume; and
(b) administering to said subject an agonist of orexin receptor 2 and/or an antagonist of orexin receptor 1.

2. The method of claim 1, wherein said antagonist of orexin receptor 1 is administered.

3. The method of claim 1, wherein said agonist of orexin receptor 2 is a adminstered.

4. The method of claim 1, wherein said agonist and/or antagonist is administered to said subject systemically.

5. The method of claim 4, wherein said agonist and/or antagonist is administered intravenously, intra-peritoneally, intramuscularly, subcutaneously or topically.

6. The method of claim 1, wherein said agonist and/or antagonist is administered to a bone target site.

7. The method of claim 6, wherein said agonist and/or antagonist is injected at said site.

8. The method of claim 6, wherein said agonist and/or antagonist is comprised in a time-release device implanted at said site.

9. The method of claim 1, wherein said subject is a human.

10. The method of claim 1, wherein said subject is a non-human animal.

11. The method of claim 10, wherein said non-human animal is a mouse, a rat, a rabbit, a dog, a cat, a horse, a monkey or a cow.

12. The method of claim 1, further comprising at least a second administration of said agonist and/or antagonist.

13. The method of claim 12, wherein said subject receives three administrations per week.

14. The method of claim 12, wherein said subject receives at least 12 administrations.

15. The method of claim 1, further comprising assessing bone mass following administration of said agonist and/or antagonist.

16. The method of claim 15, wherein assessing comprises bone imaging.

17. The method of claim 1, wherein said subject suffers from osteoporosis, bone fracture, bone loss due to trauma, rheumatoid arthritis or Paget's Disease.

18. The method of claim 1, wherein said subject does not have insomnia, obestity or diabetes.

19. A method of increasing bone growth in a subject comprising:
(a) identifying a patient in need of increased bone growth; and
(b) administering to said subject an agonist of orexin receptor 2 and/or an antagonist of orexin receptor 1.

20. The method of claim 1, wherein said antagonist of orexin receptor 1 is administered.

21. The method of claim 1, wherein said agonist of orexin receptor 2 is a adminstered.

22. The method of claim 1, wherein said agonist and/or antagonist is administered to said subject systemically.

23. The method of claim 22, wherein said agonist and/or antagonist is administered intravenously, intra-peritoneally, intramuscularly, subcutaneously or topically.

24. The method of claim 1, wherein said agonist and/or antagonist is administered to a bone target site.

25. The method of claim 24, wherein said agonist and/or antagonist is injected at said site.

26. The method of claim 24, wherein said agonist and/or antagonist is comprised in a time-release device implanted at said site.

27. The method of claim 1, further comprising at least a second administration of said agonist and/or antagonist.

28. The method of claim 12, wherein said subject receives three administrations per week.

29. The method of claim 1, further comprising assessing bone mass following administration of said agonist and/or antagonist.

30. The method of claim 15, wherein assessing comprises bone imaging.

32. The method of claim 1, wherein said subject suffers from osteoporosis, bone fracture, bone loss due to trauma, rheumatoid arthritis or Paget's Disease.

32. The method of claim 1, wherein said subject does not have insomnia, obestity or diabetes.

33. A method of increasing osteoblast number in a subject comprising:
(a) identifying a patient in need of increased osteoblast number; and
(b) administering to said subject an agonist of orexin receptor 2.

34. A method of decreasing osteoclast activity in a subject comprising:
(a) identifying a patient in need of decreased osteoclast activity; and
(b) administering to said subject an antagonist of orexin receptor 1.

35. A method of increasing bone strength in a subject comprising:
(a) identifying a patient in need of increased bone strength; and
(b) administering to said subject an agonist of orexin receptor 2 and/or an antagonist of orexin receptor 1.
